(19) United States
(12) Patent Application Publication
(10) Pub. No.: US 2024/0165213 A1
(43) Pub. Date: May 23, 2024

(54) mRNA COMPOSITION FOR PREVENTING AND TREATING CANCER

(71) Applicant: Avstera Therapeutics Corp., Malvern, PA (US)

(72) Inventors: Karthik Musunuri, Chester Springs, PA (US); Dileep Kumar, Spring City, PA (US); Andrea Facciabene, Philadelphia, PA (US)

(73) Assignee: Avstera Therapeutics Corp., Malvern, PA (US)

(21) Appl. No.: 18/515,479

(22) Filed: Nov. 21, 2023

Related U.S. Application Data
(60) Provisional application No. 63/426,882, filed on Nov. 21, 2022.

(51) Int. Cl. A61K 39/00; A61K 9/51

(52) U.S. Cl. CPC A61K 39/001129; A61K 9/5123; A61K 9/5161; A61K 2039/53; A61K 2039/55544; A61K 2039/55555

(57) ABSTRACT

The present invention provides a biologically active mRNA composition (e.g., an mRNA vaccine composition) for treating cancer and methods for using the same. In particular, the present invention relates to a biologically active mRNA composition comprising an mRNA that encodes (i) a polypeptide comprising tumor endothelial marker-1 or an immunogenic fragment thereof (“TEM1”); (ii) a polypeptide comprising tumor endothelial marker-1 or an immunogenic fragment thereof that is operatively linked to N-terminal domain of fragment C of tetanus toxoid; or (iii) a combination thereof.

### CROSS-REFERENCE TO RELATED APPLICATIONS

This application claims the priority benefit of U.S. Provisional Application No. 63/426,882, filed Nov. 21, 2022, which is incorporated herein by reference in its entirety.

### REFERENCE TO AN ELECTRONIC SEQUENCE LISTING

The contents of the following sequence listing, which has been submitted electronically, is incorporated herein by reference in its entirety:
Name of the XML File: AVS-000100US.xml;
File Size: 82,725 bytes;
Date of Creation: Nov. 20, 2023.

### FIELD

The present disclosure relates to an mRNA composition for treating cancer or tumor and methods for producing and using the same. In particular, the present disclosure relates to a composition comprising an mRNA that encodes (i) a polypeptide comprising tumor endothelial marker-1 or an immunogenic fragment thereof (“TEM1”); (ii) a polypeptide comprising tumor endothelial marker-1 or an immunogenic fragment thereof that is operatively linked to N-terminal domain of fragment C of tetanus toxoid; or (iii) a combination thereof.

### BACKGROUND

A major hurdle to advances in treating cancer is the relative lack of agents that can selectively target the cancer, while sparing normal tissue. For example, radiation therapy and surgery, which generally are localized treatments, can cause substantial damage to normal tissue in the treatment field, resulting in scarring and, in severe cases, loss of function of the normal tissue. Chemotherapy, in comparison, which generally is administered systemically, can cause substantial damage to organs such as bone marrow, mucosae, skin and the small intestine, which undergo rapid cell turnover and continuous cell division. As a result, undesirable side effects such as nausea, loss of hair and drop in blood cell count occur as a result of systemically treating a cancer patient with chemotherapeutic agents. Such undesirable side effects often limit the amount of a treatment that can be administered. Thus, cancer remains a leading cause of patient morbidity and death.

Tumor malignancies accounts for 85% cancer mortality that was responsible for 23% of all deaths in US. Current approaches for the treatment of tumor malignancies with established agents and with the new targeted agents used alone and in combination are limited, in part, by inability to deliver cytotoxic agents selectively to the tumor tissue in sufficient concentrations critical for tumor cell kill that translate into meaningful and durable responses.

Cancers metastasize through tumor vasculature, which is diverse in both its cellular and molecular compositions, exhibiting variation in the type of cells that line the vessels and their complement of cell-surface receptors. Blood vessels are one type of tumor vasculature, and archetypal blood vessels are entirely lined with endothelial cells. Tumor blood vessels also can be mosaic or lined by both endothelial and tumor cells, while other vessels are formed entirely from tumor cells. Lymphatic vessels, which also occur within several tumor types, are a second type of tumor vasculature. The lymphatic vasculature is an important route for the spreading of cancer, and animal experiments have shown a positive correlation between metastasis and the number of lymphatic vessels in and around a tumor. The development of vascular-specific tools for cancer diagnosis and/or therapy has been hindered by the paucity of targets.

Accordingly, there exists a need for improved compositions and methods for treating and/or preventing cancer.

### SUMMARY

Conventional cytotoxic therapies of cancer often suffer from a lack of specificity, leading to a poor therapeutic index and considerable toxicities to normal organs. One of the ways to overcome the disadvantages of conventional tumor therapy is the selective delivery of therapeutics to the tumor site by their conjugation to a carrier molecule specific for a tumor-associated molecular marker. Markers expressed on the tumor's vasculature represent particularly attractive targets for a site-specific pharmaco delivery due to their inherent accessibility for blood-borne agents and the various therapeutic options that they allow, ranging from intraluminal blood coagulation to the recruitment of immune cells.

To this end the present inventors have discovered that soliciting an immune response by administering an mRNA to produce antigenic proteins in vivo is particularly useful in inhibiting the growth of, suppressing the growth of, decreasing the incidence, and/or recurrence of a tumor or cancer in a subject. Thus, some aspects of the disclosure provide a composition comprising a therapeutic component or a messenger ribonucleic acid (mRNA) and a delivery vehicle. The mRNA encodes: (1) a polypeptide comprising tumor endothelial marker-1 or an immunogenic fragment thereof (“TEM1”); (ii) a polypeptide comprising tumor endothelial marker-1 or an immunogenic fragment thereof that is operatively linked to N-terminal domain of fragment C of tetanus toxoid (“TT”); or (iii) a combination thereof. In some embodiments, the mRNA comprises TEM1 mRNA, TEM1 mRNA that is linked to TT mRNA, or a combination thereof. Still in some embodiments, compositions of the disclosure are used to prevent or to treat cancer or tumor.

In one particular embodiment, said TEM1 mRNA is produced from a nucleic acid that comprises at least 80% homology to SEQ ID NO:1. Still in other embodiments, said mRNA that encodes TEM1 comprises at least one nucleoside that is substituted with a modified nucleoside analog thereof. In one particular embodiment, said modified nucleoside analog comprises pseudouridine or N1-methylpseudouridine.

Yet in other embodiments, said delivery vehicle comprises:
a lipid nanoparticle;
protamine;
a positively charged oil-in-water cationic nanoemulsion;
a chemically modified dendrimer complexed with a lipid;
cationic polymer comprising a lipid component;
polysaccharide; or
a cationic lipid, cholesterol, and optionally (i) a neutral lipid, (ii) a polyethylene-lipid, or (iii) a combination thereof.

In further embodiments, said lipid nanoparticle comprises cationic lipid, cholesterol, a neutral lipid, a polyethylene-lipid, or a combination thereof.

Still in other embodiments, said delivery vehicle is complexed with said mRNA. Yet in other embodiments, said mRNA is encapsulated within said delivery vehicle.

Another aspect of the invention provides a method for producing a composition comprising a delivery vehicle and an active therapeutic component or an mRNA disclosed herein. Typically, the mRNA encodes:
(i) a polypeptide comprising tumor endothelial marker-1 or an immunogenic fragment thereof (“TEM1”);
(ii) a polypeptide comprising tumor endothelial marker-1 or an immunogenic fragment thereof that is operatively linked to an N-terminal domain of fragment C of tetanus toxoid (“TEM1-TT”); or
(iii) a combination thereof.
   The method for producing the composition disclosed herein includes:
(a) producing said mRNA by transcription using a plasmid comprising a nucleic acid sequence that encodes said mRNA; and
(b) combining said mRNA with said delivery vehicle to produce said composition.

In some embodiments, said mRNA that encodes TEM1 is produced from a nucleic acid that comprises at least about 80%, typically at least about 85%, often at least about 90%, more often at least about 95%, still more often at least about 97%, yet more often at least about 98%, further more often at least about 99%, and most often 100% homology to SEQ ID NO:1, 4, 7, or 10. Still in other embodiments, said mRNA is produced from a plasmid construct having at least about 80% identity, 85% identity, 90% identity, 91% identity, 92% identity, 93% identity, 94% identity, 95% identity, 96% identity, 97% identity, 98% identity, 99% identity, or 100% identity to SEQ ID NO: 16, 17, 18, or 19. Alternatively, the mRNA of the invention comprises at least about 80% identity, about 85% identity, 90% identity, 91% identity, 92% identity, 93% identity, 94% identity, 95% identity, 96% identity, 97% identity, 98% identity, 99% identity, or 100% identity to the mRNA sequence of SEQ ID NO:2, 5, 8, or 11. Yet in another embodiment, the mRNA of the invention encodes protein having at least about 80% identity, about 85% identity, 90% identity, 91% identity, 92% identity, 93% identity, 94% identity, 95% identity, 96% identity, 97% identity, 98% identity, 99% identity, or 100% identity to SEQ ID NO: 3, 6, 9, or 12.

Still in other embodiments, said step of producing said mRNA comprises:
(a) producing said mRNA using a nucleic acid that comprises at least about 80% identity, about 85% identity, 90% identity, 91% identity, 92% identity, 93% identity, 94% identity, 95% identity, 96% identity, 97% identity, 98% identity, 99% identity, or 100% identity to SEQ ID NO:1, 4, 7, or 10; and
(c) isolating said mRNA.

In some embodiments, the produced mRNA comprises at least about 80%, at least about 85%, 90% identity, 91% identity, 92% identity, 93% identity, 94% identity, 95% identity, 96% identity, 97% identity, 98% identity, 99% identity, or 100% identity to the mRNA sequence of SEQ ID NO: 2, 5, 8, or 11. Yet in another embodiment, the produced mRNA encodes a protein having at least about 80%, at least about 85%, 80% identity, 85% identity, 90% identity, 91% identity, 92% identity, 93% identity, 94% identity, 95% identity, 96% identity, 97% identity, 98% identity, 99% identity, or 100% identity to SEQ ID NO: 3, 6, 9, or 12.

Yet in other embodiments, said mRNA encoding TEM1 comprises at least one nucleoside that is substituted with a modified nucleoside analog thereof. In some instances, said modified nucleoside analog comprises pseudouridine or N1-methylpseudouridine.

In further embodiments, said step (b) of producing said composition comprises complexing said mRNA with said delivery vehicle. In other embodiments, said step (b) of producing said composition comprises encapsulating said active therapeutic component within said delivery vehicle.

Still another aspect of the invention provides a method for of inhibiting the growth of, suppressing the growth of, decreasing the incidence, and/or recurrence of a tumor in a subject, said method comprising administering to said subject a composition comprising a delivery vehicle and an active therapeutic or mRNA component, wherein said composition or active therapeutic component comprises tumor endothelial marker-1 mRNA (“TEM1 mRNA), TEM1 mRNA linked to N-terminal domain of fragment C of tetanus toxoid mRNA (“TT mRNA), or a combination thereof.

In some embodiments, said mRNA comprises said TEM1 mRNA. Yet in other embodiments, said active therapeutic component comprises said TEM1 mRNA linked to TT mRNA, wherein TT mRNA comprises a N-terminal domain of fragment C of tetanus toxoid.

Still in other embodiments, said TEM1 mRNA encodes a polypeptide comprising tumor endothelial marker-1 or an immunogenic fragment thereof.

In further embodiments, said TT mRNA encodes a polypeptide having at least about 80%, at least about 85%, 90% identity, 91% identity, 92% identity, 93% identity, 94% identity, 95% identity, 96% identity, 97% identity, 98% identity, 99% identity, or 100% identity to N-terminal domain of fragment C of tetanus toxoid.

Yet in other embodiments, cancer or tumor that is treated, inhibited, suppressed, decreased, or prevented from recurrence comprises lung cancer or tumor, melanoma, breast cancer or tumor, kidney cancer or tumor, cervical cancer or tumor, head and neck cancer or tumor, breast cancer or tumor, an ano-genital cancer or tumor. The cancer may be a melanoma, a sarcoma, a carcinoma, a lymphoma, a leukemia, a mesothelioma, a glioma, a germ cell tumor, a choriocarcinoma, a pancreatic cancer, an ovarian cancer, a gastric cancer, a carcinomatous lesion of the pancreas, pulmonary adenocarcinoma, colorectal adenocarcinoma, pulmonary squamous adenocarcinoma, gastric adenocarcinoma, an ovarian surface epithelial neoplasm (e.g., a benign, proliferative or malignant variety thereof), an oral squamous cell carcinoma, a non small-cell lung carcinoma, an endometrial carcinoma, a bladder cancer, a prostate carcinoma, or an acute myelogenous leukemia (AML), a colon cancer, a lung cancer, a germ cell tumor, a uterine cancer, a thyroid cancer, a hepatocellular carcinoma, a liver cancer, a renal cancer, a Kaposi's sarcoma, or a sarcoma. The cancer may be another carcinoma or sarcoma.

Some aspects of the invention provide producing mRNA for use as a vaccine. Construction of mRNA vaccines typically involves the insertion of the encoded oligonucleotide in a DNA template from where the mRNA is transcribed in vitro, isolated and used as an active vaccine component. The mRNA when administered to a subject, is incorporated into cells of the subject and produces the desired antigen to stimulate the subject's immune response. Unlike DNA, mRNA only needs to reach the cytosol, where it can be transcribed into the antigen in vivo, using the cell machinery. In general, any cells can be used to produce mRNA. In some embodiments, E. Coli, or other cells that are known to one skilled in the art is used to produce mRNA. Production of mRNA for vaccine use is well known to one skilled in the art, for example, production of SARS-Cov-mRNA vaccines are well known, and a similar process can be used to produce mRNA vaccine of the present invention.

One skilled in the art having read the present disclosure can readily design and produce other derivatives or modified nucleotide sequences of mRNA disclosed herein. Such derivatives or modified nucleotide sequences of mRNA can be used to produce various mRNA vaccines of the invention, for example, by transfecting such modified mRNA to any suitable cells or organisms to produce the desired mRNA. See, for example, Maruggi et al., Mol Ther, 2019, 27(4), pp. 757-772.

Another aspect of the disclosure provides a composition for inducing an immune response against a tumor endothelial marker-1 (TEM1) protein, said composition comprising a delivery vehicle and a TEM1 mRNA that comprises a nucleotide sequence encoding a TEM1 protein or an immunogenic fragment thereof. In some embodiments, said delivery vehicle comprises a nanoparticle, a nanosome, a liposome, a biodegradable polymer complex, or a combination thereof. Still in other embodiments, said delivery vehicle comprises: a lipid nanoparticle (LNP); protamine; a positively charged oil-in-water cationic nanoemulsion; a chemically modified dendrimer complexed with a lipid; a cationic polymer comprising a lipid component; a polysaccharide; or a cationic lipid, cholesterol, and optionally (i) a neutral lipid, (ii) a polyethylene-lipid, or (iii) a combination thereof. In one particular embodiment, said delivery vehicle comprises said lipid nanoparticle (LNP). In some instances, said LNP comprises cationic lipid, an ionizable cationic lipid, a sterol, a neutral lipid, a polyethylene-lipid, a polyethylene glycol (PEG)-modified lipid, a polymer-lipid conjugate, or a combination thereof.

Yet in further embodiments, said composition further comprises an adjuvant. In some embodiments, said adjuvant comprises a TT mRNA that is operatively linked to said TEM1 mRNA, and wherein said TT mRNA comprises a nucleotide sequence that encodes a tetanus toxoid or an immunogenic fragment thereof. Still in other embodiments, said TT mRNA has a nucleotide sequence having at least 80% identity to SEQ ID NO: 23 or a portion of SEQ ID NO: 23 that produces an immunogenic fragment of tetanus toxoid. In further embodiments, said TEM1 mRNA that is operatively linked to said TT mRNA encodes a polypeptide comprising TEM1 protein or an immunogenic fragment thereof that is operatively linked to N-terminal domain of fragment C of tetanus toxoid or an immunogenic fragment thereof. Yet in other embodiments, said TEM1 mRNA that is operatively linked to said TT mRNA has a nucleotide sequence having at least 80% identity to SEQ ID NO: 5. In other embodiments, said TEM1 mRNA comprises a nucleic acid sequence that is at least 80% identity to SEQ ID NO: 2 or a portion of SEQ ID NO: 2 that produces said immunogenic fragment of TEM1 protein. Still yet in other embodiments, said TEM1 mRNA comprises a nucleotide sequence encoding said immunogenic fragment of TEM1 protein. In some instances, said immunogenic fragment of TEM1 protein comprises an amino acid sequence having at least 95% identity to amino acid residue 516-530, 511-525, 696-710, or a combination thereof of SEQ ID NO: 3.

Yet in other embodiments, said TEM1 mRNA comprises at least one modified nucleoside. In some instances, at least one uridine is replaced with said modified nucleoside. Still in other instances, said modified nucleoside is N1-methyl-pseudouridine (m1ψ).

In other embodiments, said TEM1 mRNA is present in an amount within a range of from about 1 μg to about 100 μg per dose.

Another aspect of the disclosure provides a method for producing a composition or medical preparation comprising a biologically active mRNA, wherein said biologically active mRNA comprises a TEM1 mRNA nucleotide sequence encoding a tumor endothelial marker-1 (TEM1) protein or an immunogenic fragment thereof, said method comprising:
(a) producing said biologically active mRNA by in vitro transcription using a plasmid comprising a nucleic acid sequence that encodes said biologically active mRNA;
(b) isolating said biologically active mRNA; and
(b) combining said isolated biologically active mRNA with a delivery vehicle to produce said composition or medical preparation.

In some embodiments, said delivery vehicle comprises a nanoparticle, a nanosome, a liposome, a biodegradable polymer complex, or a combination thereof. Still in other embodiments, said delivery vehicle comprises: a lipid nanoparticle (LNP); protamine; a positively charged oil-in-water cationic nanoemulsion; a chemically modified dendrimer complexed with a lipid; a cationic polymer comprising a lipid component; a polysaccharide; or a cationic lipid, cholesterol, and optionally (i) a neutral lipid, (ii) a polyethylene-lipid, or (iii) a combination thereof. In one particular embodiment, said delivery vehicle comprises said lipid nanoparticle (LNP). In another particular said LNP comprises cationic lipid, an ionizable cationic lipid, a sterol, a neutral lipid, a polyethylene-lipid, a polyethylene glycol (PEG)-modified lipid, a polymer-lipid conjugate, or a combination thereof.

Yet in other embodiments, said biologically active mRNA further comprises a TT mRNA that is operatively linked to said TEM1 mRNA, and wherein said TT mRNA comprises a nucleotide sequence that encodes a tetanus toxoid or an immunogenic fragment thereof. In some instances, said TT mRNA has a nucleotide sequence having at least 80% identity to SEQ ID NO: 23 or a portion of SEQ ID NO: 23 that produces an immunogenic fragment of tetanus toxoid. Still in other embodiments, said TEM1 mRNA that is operatively linked to said TT mRNA encodes a polypeptide comprising TEM1 protein or an immunogenic fragment thereof that is operatively linked to N-terminal domain of fragment C of tetanus toxoid or an immunogenic fragment thereof. Yet in other embodiments, said TEM1 mRNA that is operatively linked to said TT mRNA has a nucleotide sequence having at least 80% identity to SEQ ID NO: 5. In further embodiments, said TEM1 mRNA comprises a nucleic acid sequence that is at least 80% identity to SEQ ID NO: 2 or a portion of SEQ ID NO: 2 that produces said immunogenic fragment of TEM1 protein. In another embodiment, said TEM1 mRNA comprises a nucleotide sequence encoding said immunogenic fragment of TEM1 protein. In some instances, said immunogenic fragment of TEM1 protein comprises an amino acid sequence having at least 95% identity to amino acid sequence of SEQ ID NO: 20, 21, or 22.

Yet in other embodiments of the method, said TEM1 mRNA comprises at least one modified nucleoside. In other embodiments, at least one uridine is replaced with said modified nucleoside. Still in other embodiments, said modified nucleoside is N1-methyl-pseudouridine (mli).

Still another aspect of the disclosure provides a method for eliciting an immune response against TEM1 protein in a subject comprising administering to the subject a therapeutically effective amount of a composition disclosed herein. In some embodiments, mRNA comprises a nucleotide sequence encoding said immunogenic fragment of TEM1 protein. In one particular embodiment, said immunogenic fragment of TEM1 protein comprises an amino acid sequence having at least 95% identity to amino acid sequence of SEQ ID NO: 19, 20, 21, or a combination thereof.

Yet another aspect of the disclosure provides a method for of inhibiting the growth of, suppressing the growth of, decreasing the incidence, and/or recurrence of a tumor or a cancer cell in a subject, said method comprising administering to said subject a therapeutically effective amount of a composition disclosed herein.

Still another aspect of the disclosure provides a method for treating a tumor or cancer in a subject comprising: administering to a subject in need of such a treatment a therapeutically effective amount of a composition disclosed herein. In some embodiments, said cancer or tumor comprises lung cancer or tumor, melanoma, breast cancer or tumor, kidney cancer or tumor, cervical cancer or tumor, head and neck cancer or tumor, breast cancer or tumor, an ano-genital cancer or tumor. Still in other embodiments, said cancer comprises a melanoma, a sarcoma, a carcinoma, a lymphoma, a leukemia, a mesothelioma, a glioma, a germ cell tumor, a choriocarcinoma, a pancreatic cancer, an ovarian cancer, a gastric cancer, a carcinomatous lesion of the pancreas, pulmonary adenocarcinoma, colorectal adenocarcinoma, pulmonary squamous adenocarcinoma, gastric adenocarcinoma, an ovarian surface epithelial neoplasm (e.g., a benign, proliferative or malignant variety thereof), an oral squamous cell carcinoma, a non small-cell lung carcinoma, an endometrial carcinoma, a bladder cancer, a prostate carcinoma, an acute myelogenous leukemia (AML), a colon cancer, a lung cancer, a germ cell tumor, a uterine cancer, a thyroid cancer, a hepatocellular carcinoma, a liver cancer, a renal cancer, a Kaposi's sarcoma, or a sarcoma.

### BRIEF DESCRIPTION OF THE DRAWINGS

FIG. 1 is western blot showing the results of TEM1 and TEM1-TT protein expression in transfected HEK 293 cells.

FIG. 2 shows RNA expression analysis from plasmid DNA transfection of TEM1 and TEM1-TT.

FIG. 3 shows calculation of the volume of components needed to prepare the required number of reactions.

FIG. 4 is a schematic representation of TEM1 mRNA used in the disclosure.

FIG. 5 is a schematic representation of TEM1-TT mRNA used in the disclosure.

FIG. 6 shows integrity and quality of mRNA using agarose gel electrophoresis.

FIG. 7 shows PCR results with the respective amplicon size confirming the integrity and specificity of in vitro synthesized TEM1 and TEM1-TT mRNA.

### DETAILED DESCRIPTION

Cancer still remains one of the leading causes of death. Currently, cancer is responsible for more than 20% of all deaths in the U.S. Of those, tumor malignancies account for more than 85% of cancer mortality. Current approaches for the treatment of tumor malignancies with established agents and with the new targeted agents used alone and in combination are limited, in part, by inability to deliver cytotoxic agents selectively to the tumor tissue in sufficient concentrations for tumor cell kill that translate into meaningful and durable responses.

Provided herein are compositions comprising a therapeutically active agent that selectively targets tumor cells. In one particular aspect of the disclosure, a composition comprising a messenger ribonucleic acid (mRNA) and a delivery vehicle is provided. Compositions comprising an mRNA can safely and effectively direct the body's cellular machinery to produce a protein of interest to elicit body's own immune response to tumor cells. Accordingly, mRNA of the present disclosure can be used to induce a subject's own immune response against tumor cells. The composition comprising an mRNA disclosed herein can be used to treat cancer or to prevent occurrence or recurrence of cancer.

Some aspects of the disclosure provide a composition comprising an mRNA and methods for producing and using the same. In one particular aspect of the disclosure, the composition disclosed herein is used to (i) immunize a subject against a tumor, (ii) inhibit tumor growth, (iii) inhibit tumor recurrence, and/or (iv) treat, suppress the growth of, or decrease the incidence of a tumor. In one particular embodiment, the composition disclosed herein is used to overcome tolerance to a tumor vasculature marker (TEM1).

In one particular aspect of the disclosure, a composition comprising an mRNA (e.g., a vaccine) and a delivery vehicle is provided. The mRNA is a biologically active mRNA. In some embodiments, the biologically active mRNA component comprises a messenger ribonucleic acid (mRNA) encoding:
(1) a polypeptide comprising tumor endothelial marker-1 or an immunogenic fragment thereof (“TEM1”);
(ii) a polypeptide comprising tumor endothelial marker-1 or an immunogenic fragment thereof (“TEM1”) that is operatively linked to N-terminal domain of fragment C of tetanus toxoid (“TT”); or
(iii) a combination thereof.

It should be appreciated that the biologically active composition of the invention can be used as a vaccine to prevent a tumor or cancer from occurring or reoccurring in a subject. In addition, the biologically active composition of the disclosure can be used to treat a tumor or cancer. Accordingly, unless the context requires otherwise, the term “vaccine” means use of the biologically active composition comprising an mRNA of the disclosure to treat, prevent, and/or to ameliorate occurrence of a tumor or cancer.

The composition as provided herein, comprise at least one mRNA or polynucleotide having an open reading frame encoding TEM1 or an immunogenic fragment thereof. The term “nucleic acid” includes any compound and/or substance that comprises a polymer of nucleotides (nucleotide monomer). These polymers are referred to as polynucleotides. Thus, the terms “nucleic acid” and “polynucleotide” are used interchangeably.

Nucleic acids can include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. One of ordinary skill in the art will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.” Moreover, because “U” is not accepted in the current .ST26 sequence listing program, all “U” in sequence listings accompanying this disclosure is substituted with “T.” The basic components of an mRNA molecule typically include at least one coding region, a 5′-untranslated region (UTR), a 3′-UTR, a 5′-cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

In some embodiments, an mRNA of the disclosure encodes TEM1 protein or an immunogenic fragment thereof (i.e., an antigenic polypeptide thereof). As used herein, the term “an immunogenic fragment thereof” refers to a portion of the protein that elicits an immune response. Such fragments of TEM1 protein and tetanus toxoid are well known to one of ordinary skilled in the art or can be readily determined using standard technics and methods known to one skilled in the art. See, for example, Facciponte et al., J Clin Invest. 2014, 124(4), pp. 1497-1511. Briefly, a peptide of 15-mer, 20-mer, or 25-mer can be synthesized starting from amino acid residue 1, until the end, e.g., for 15-mer: 1-15, 2-16, 3-17, 4-18, . . . , 749-763, 750-764, and 751-765. These peptides can then be administered to, e.g., an animal model to determine CD8+ and CD4+ T-cell response to determine immunogenic fragments (i.e., antigenic polypeptides). In some embodiments, the mRNA polynucleotide of the disclosure encodes TEM1 protein residue 516-530 (SEQ ID NO: 20), 511-525 (SEQ ID NO: 21), and/or 696-710 (SEQ ID NO: 22).

Still in other embodiments, an mRNA of the disclosure encodes a polypeptide comprising TEM1 protein or an immunogenic fragment thereof that is operatively linked to N-terminal domain of fragment C of tetanus toxoid (“TT”) or an immunogenic fragment thereof. As used herein, the term “tetanus toxoid” or “TT” refers to N-terminal domain of fragment C of tetanus toxoid. In some embodiments, mRNA encodes a polypeptide comprising TEM1 protein or an immunogenic fragment thereof that is fused or linked to the first domain of the C fragment of the TT sequence (i.e., TT 865-1,120) at its carboxyl terminal (SEQ ID NO: 23).

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art. Exemplary services include, but are not limited to, services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares 99% or less, 98% or less, 97% or less, 96% or less, 95% or less, 94% or less, 93% or less, 92% or less, 91% or less, 90% or less, 85% or less, 80% or less, or 75% or less sequence identity to a naturally-occurring or wild-type sequence (e.g., a naturally-occurring or wild-type mRNA sequence encoding a polypeptide or protein of interest, e.g., TEM1 or immunogenic fragment thereof and/or N-terminal domain of fragment C of TT or an immunogenic fragment thereof).

In some embodiments, a codon-optimized sequence shares about 65% or more, 70% or more, 75% or more, 80% or more, 85% or more, 90% or more, 91% or more, 92% or more, 93% or more, 94% or more, 95% or more, 96% or more, 97% or more, 98% or more, and 99% or more sequence identity to a naturally-occurring sequence or a wild-type sequence (e.g., a naturally-occurring or wild-type mRNA sequence encoding TEM1 or an immunogenic fragment thereof and/or N-terminal domain of fragment C of TT or an immunogenic fragment thereof). In some embodiments, a codon-optimized sequence shares from about 65% to about 75%, or about 80% sequence identity to a naturally-occurring sequence or wild-type sequence (e.g., a naturally-occurring or wild-type mRNA sequence encoding TEM1 or an immunogenic fragment thereof and/or N-terminal domain of fragment C of TT or an immunogenic fragment thereof). When referring to a numerical value, the terms “about” and “approximately” are used interchangeably herein and refer to being within an acceptable error range for the particular value as determined by one of ordinary skill in the art. Such a value determination will depend at least in part on how the value is measured or determined, e.g., the limitations of the measurement system, i.e., the degree of precision required for a particular purpose. For example, the term “about” can mean within 1 or more than 1 standard deviation, per the practice in the art. Alternatively, the term “about” when referring to a numerical value can mean±20%, typically ±10%, often ±5% and more often ±1% of the numerical value. In general, however, where particular values are described in the application and claims, unless otherwise stated, the term “about” means within an acceptable error range for the particular value, typically within one standard deviation.

Yet in other embodiments a codon-optimized mRNA can be one in which the levels of G/C are enhanced. The G/C-content of nucleic acid molecules may influence the stability of the mRNA. mRNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater mRNA stability without changing the resulting amino acid. The approach is limited to coding regions of the mRNA.

In some embodiments, an immunogenic fragment of TEM1 or TT protein is about 10 amino acids or longer, often about 15 amino acids or longer. Still in other embodiments, an immunogenic fragment of TEM1 or TT protein is about 50 amino acids or less. Polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. The term “polypeptide” may also apply to amino acid polymers in which at least one amino acid residue is an artificial chemical analogue of a corresponding naturally-occurring amino acid.

A “polypeptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity, at least about 85%, at least about 90%, at least about 91%, at least about 92%, at least about 93%, at least about 94%, at least about 95%, at least about 96%, at least about 97%, at least about 98%, or at least about 99% identity with a native sequence or a reference sequence.

In some embodiments “variant mimics” are provided. A “variant mimic” contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic. For example, phenylalanine may act as an inactivating substitution for tyrosine, or alanine may act as an inactivating substitution for serine.

“Orthologs” refers to genes in different species that evolved from a common ancestral gene by speciation. Normally, orthologs retain the same function in the course of evolution. Identification of orthologs is important for reliable prediction of gene function in newly sequenced genomes.

“Analogs” is meant to include polypeptide variants that differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding TEM1 or an immunogenic fragment thereof or TT or an immunogenic fragment thereof containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini and any combination(s) thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide-based or polynucleotide-based molecules.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of at least 5, at least 10, at least 15, at least 20, at least 25, at least 30, at least 35, at least 40, at least 45, at least 50, at least 75, or at least 100 amino acids. In another example, any protein that includes a stretch of 10, 15, 20, 25, 30, 35, 40, 45, or 50 (contiguous) amino acids that are 40%, 50%, 60%, 70%, 80%, 90%, 95%, or 100% identical to any of the sequences described herein can be utilized in accordance with the disclosure. In some embodiments, a polypeptide includes 1, 2, 3, 4, 5, 6, 7, 8, 9, 10, or more mutations as shown in any of the sequences provided herein or referenced herein. In another example, any protein that includes a stretch of 5, 10, 15, 20, 25, 30, 35, 40, 45, or 50 amino acids that are greater than 80%, 90%, 95%, or 100% identical to any of the sequences described herein can be utilized in accordance with the disclosure.

In one embodiment, TEM1 mRNA, i.e., mRNA that encodes a polypeptide comprising tumor endothelial marker-1 or an immunogenic fragment thereof, is encoded by a nucleic acid sequence comprising at least about 80%, typically at least about 85%, often at least about 90% and most often at least about 95% sequence identity to SEQ ID NO: 1, 4, 7, 10, 16, 17, or 19, or a cDNA sequence thereof. For the sake of brevity and clarity, throughout this disclosure, unless context requires otherwise, all references to nucleic acid sequence also includes cDNA sequence thereof.

The term “percent (%) sequence identity” as used herein is used to denote sequences which when aligned have similar (identical or conservatively replaced) amino acids in like positions or regions, where identical or conservatively replaced amino acids are those which do not alter the activity or function of the protein as compared to the starting protein. For example, two amino acid sequences with at least 85% identity to each other have at least 85% similar (identical or conservatively replaced residues) in a like position when aligned optimally allowing for up to 3 gaps, with the proviso that in respect of the gaps a total of not more than 15 amino acid residues is affected. Percent sequence identity may be calculated by determining the number of residues that differ between a peptide encompassed by the present invention and a reference peptide, such as SEQ ID NO: 1, taking that number and dividing it by the number of amino acids in the reference peptide, multiplying the result by 100, and subtracting that resulting number from 100. The degree of sequence identity may be determined using methods well known in the art (see, for example, Wilbur, W. J. et al., Proc. Natl. Acad. Science USA, 1983, 80, 726-730 and Myers E. et al., Comput. Appl. Biosci., 1988, 4, 11-17. One program which may be used in determining the degree of similarity is the MegAlign Lipman-Pearson one pair method (using default parameters) which can be obtained from DNAstar Inc, 1128, Selfpark Street, Madison, Wisconsin, 53715, USA as part of the Lasergene system. Another program, which may be used, is Clustal W. This is a multiple sequence alignment package developed by Thompson et al. (Nucleic Acids Research, 1994, 22(22), 4673-4680) for DNA or protein sequences. This tool is useful for performing cross-species comparisons of related sequences and viewing sequence conservation. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen. Evolutionary relationships can be seen via viewing Cladograms or Phylograms.

The term “fragment” means a nucleic acid sequence having one or more (e.g., several) nucleic acids deleted from the reference nucleic acid sequence. In one aspect, a fragment contains at least about 80%, typically at least 85%, often at least 90%, at least 91%, at least 92%, at least 93%, at least 94%, at least about 95%, at least 96%, at least 97%, at least about 98%, and more often at least 99% nucleic acid sequences.

SEQ ID NOS:1-3 are human TEM1 DNA sequence, human TEM1 mRNA sequence, and human TEM1 protein sequence, respectively. SEQ ID NOS: 4-6 are human TEM1 linked to N-terminal domain of fragment C of tetanus toxoid (TT) DNA sequence, human TEM1-TT mRNA sequence, and human TEM1-TT protein sequence, respectively. The corresponding DNA that encodes mouse TEM1 mRNA, mouse TEM1 mRNA sequence, mouse TEM1 protein sequence, DNA nucleotide that encodes mouse TEM1-TT, mouse TEM1-TT mRNA sequence, and mouse TEM-TT protein are shown in SEQ ID NOS: 7-12, respectively. In some embodiments, nucleotide sequence 1, 4, 7, or, 10, or a variation thereof, or a fragment thereof, is used to produce the mRNA. The produced mRNA can be mRNA sequence having SEQ ID NOS: 2, 5, 8, or 11, or a variation thereof, or a fragment thereof, respectively. Such mRNA encodes protein having SEQ ID NOS: 3, 6, 9, or 12, or a variation or a fragment thereof, respectively.

In some embodiment, nucleic acid sequence encoding TEM1 mRNA also includes 5′-UTR, such as SEQ ID NO: 13, that includes Kozak consensus sequence, the last 6 nucleotides. Yet in other embodiments, nucleic acid sequence encoding TEM1 mRNA also includes 3′-UTR, such as SEQ ID NO: 14. Still in other embodiments, nucleic acid sequence encoding TEM1 mRNA also includes poly-A tail optionally with Esp 3I restriction site, such as SEQ ID NO: 15, where the last 7 nucleotide corresponds to Esp 3I restriction site.

In further embodiments, the TEM1 mRNA of the invention is encoded by a nucleic acid sequence comprising SEQ ID NO: 1, 13, 14, and 15. Such nucleic acid sequence is represented by SEQ ID NO: 16. In some embodiments, SEQ ID NO: 16 is used as a plasmid DNA to produce TEM1 protein using a host cell.

It should be appreciated some of the components of SEQ ID NO: 5, such as 5′-UTR, Kozak Seq, Linker Sequence, 3′-UTR, Poly-A tail, and Esp3I Restriction Site are optional or maybe replaced with other components.

Yet still in other embodiments, the mRNA that encodes TEM1 comprises at least one nucleoside that is substituted with a modified nucleoside analog thereof. As used herein, the term “modified nucleoside” refers to a nucleoside comprising at least one modification compared to naturally occurring RNA nucleosides. Such modification may be at the sugar moiety and/or at the nucleobases. Exemplary modified nucleosides include, but are not limited to, pseudouridine, N1-methylpseudouridine, a modified nucleoside, wherein the modified nucleoside is m5C, m5U, m6A, s2U, Ψ, or 2′-O-methyl-U and other modified nucleosides known to one skilled in the art. See, for example, Yates et al., Antiviral Res. 2019, 162, pp. 5-21; and U.S. Pat. Nos. 8,993,738; and 9,321,799, all of which are incorporated herein by reference in their entirety.

Still in other embodiments, the mRNA encodes a polypeptide comprising tumor endothelial marker-1 or an immunogenic fragment thereof (“TEM1”) that is operatively linked to tetanus toxoid or an N-terminal domain of fragment C of tetanus toxoid (collectively “TT”). In one particular embodiment, mRNA that encodes a polypeptide comprising TEM1 linked TT, i.e., TEM1 mRNA-TT mRNA, is encoded by a nucleic acid sequence comprising at least about 80%, typically at least about 85%, often at least about 90% and most often at least about 95% sequence identity to SEQ ID NO: 17. As stated above, unless context requires otherwise, all references to nucleic acid sequence also includes cDNA sequence thereof.

It should be appreciated some of the components of SEQ ID NO: 17, such as 5′-UTR, Kozak Seq, Linker Sequence, 3′-UTR, Poly-A tail, and Esp3I Restriction Site are optional or maybe replaced with other components.

The corresponding mouse TEM1 mRNA, i.e., mRNA that encodes a polypeptide comprising mouse tumor endothelial marker-1 or an immunogenic fragment thereof, is encoded by a nucleic acid sequence comprising at least about 80%, typically at least about 85%, often at least about 90% and most often at least about 95% sequence identity to SEQ ID NO:18.

It should be appreciated some of the components of SEQ ID NO: 18, such as 5′-UTR, Kozak Seq, Linker Sequence, 3′-UTR, Poly-A tail, and Esp3I Restriction Site are optional or maybe replaced with other components.

Still in other embodiments, the mRNA encodes a mouse polypeptide comprising tumor endothelial marker-1 or an immunogenic fragment thereof (“TEM1”) that is operatively linked to tetanus toxoid or an N-terminal domain of fragment C of tetanus toxoid (collectively “TT”). In one particular embodiment, mRNA that encodes a mouse polypeptide comprising TEM1 linked TT, i.e., mouse TEM1 mRNA-TT mRNA, is encoded by a nucleic acid sequence comprising at least about 80%, typically at least about 85%, often at least about 90% and most often at least about 95% sequence identity to SEQ ID NO: 19. As stated above, unless context requires otherwise, all references to nucleic acid sequence also includes cDNA sequence thereof.

It should be appreciated some of the components of SEQ ID NO: 8, such as 5′-UTR, Kozak Seq, Linker Sequence, 3′-UTR, Poly-A tail, and Esp3I Restriction Site are optional or maybe replaced with other components.

In further embodiments, the mRNA encodes amino acid sequence having at least about 90%, typically at least about 95%, and often at least about 98% identity to the amino acid sequence of SEQ ID NO: 3:

Yet in other embodiments, mRNA encodes amino acid sequence having at least about 90%, typically at least about 95%, and often at least about 98% identity to the amino acid sequence of SEQ ID NO: 6.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl pseudouridine, or another modified nucleoside.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art is well aware that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

In certain embodiments, “pseudouridine” refers, in another embodiment, to m¹acp³Ψ (1-methyl-3-(3-amino-3-carboxypropyl) pseudouridine. In another embodiment, the term refers to m¹Ψ (1-methylpseudouridine). In another embodiment, the term refers to Ψm (2′-O-methylpseudouridine. In another embodiment, the term refers to m⁵D (5-methyldihydrouridine). In another embodiment, the term refers to m³Ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

Other substantial modifications in function or immunological identity of the target polypeptide maybe accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another. Such substituted residues may be introduced into regions of the target polypeptide that are homologous with other immunogenic regions of the same class or subclass, or into the non-homologous regions of the molecule. For example, any cysteine residues not involved in maintaining the proper conformation of target polypeptide also may be substituted, generally with serine, to improve the oxidative stability of the molecule and prevent aberrant crosslinking. Suitable mRNA for such a peptide or amino acid residue substitution are well known to one skilled in the art.

In one embodiment, the invention includes a nucleoside-modified nucleic acid molecule. In one embodiment, the nucleoside-modified nucleic acid molecule encodes TEM1 mRNA or TEM1 mRNA that is operatively linked to TT mRNA (“TEM1 mRNA-TT mRNA”). In certain embodiments, the nucleoside-modified nucleic acid molecule encodes TEM1 mRNA or TEM1 mRNA-TT mRNA that induces an adaptive immune response.

The nucleotide sequences encoding TEM1 mRNA or TEM1 mRNA-TT mRNA, as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the nucleotide sequences recited herein and encode TEM1 mRNA or TEM1 mRNA-TT mRNA.

A nucleotide sequence that is substantially homologous to a nucleotide sequence encoding TEM1 mRNA or TEM1 mRNA-TT mRNA can typically be isolated from a producer organism based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

Further, the scope of the invention includes mRNA that encode protein sequences that are substantially homologous to the amino acid sequences recited herein and preserve the immunogenic function of the original amino acid sequence (e.g., SEQ ID NOs: 9 and 10).

As used herein, an amino acid sequence is “substantially homologous” to any of the amino acid sequences described herein when its amino acid sequence has a degree of identity with respect to the amino acid sequence of at least 60%, of at least 65%, of at least 70%, of at least 65%, of at least 80%, of at least 85%, of at least 90%, of at least 95%, of at least 96%, of at least 97%, of at least 98%, or of at least 99%. The identity between two amino acid sequences can be determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In one embodiment, the invention relates to a construct or a vector comprising a nucleotide sequence encoding TEM1 mRNA or TEM1 mRNA-TT mRNA. In one embodiment, the construct comprises a plurality of nucleotide sequences encoding a plurality of mRNA. For example, in certain embodiments, the construct encodes TEM1 mRNA, or TEM1 mRNA-TT mRNA, or both.

In another particular embodiment, the construct is operatively bound to a translational control element. The construct can incorporate an operatively bound regulatory sequence for the expression of the nucleotide sequence of the invention, thus forming an expression cassette.

The nucleic acid sequences coding for the mRNA can be obtained using recombinant methods known in the art, such as, for example by producing the nucleic acid synthetically.

The nucleic acid can be cloned into a number of types of vectors. For example, the nucleic acid can be cloned into a vector including, but not limited to a plasmid, a phagemid, a phage derivative, an animal virus, a PCR-generated linear DNA sequence, and a cosmid. Vectors of particular interest include expression vectors, replication vectors, probe generation vectors, sequencing vectors and vectors optimized for in vitro transcription.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, carbohydrates, peptides, cationic polymers, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Generally, chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology, 1991, 5, pp. 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the mRNA of the present invention, in order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the invention.

In one embodiment, the composition of the invention comprises in vitro transcribed mRNA. In one embodiment, the composition of the invention comprises mRNA encoding TEM1 protein or TEM1 protein that is operatively linked to TT, or a combination thereof.

In one embodiment, nucleic acid sequences that encodes the mRNA can be introduced to a cell to produce the mRNA. The mRNA is produced by in vitro transcription using a plasmid DNA or nucleic acid sequence template (e.g., SEQ ID NOs: 5 or 8). Nucleic acids or DNA of TEM1 from any source, including synthetically produced nucleic acid sequences, can be directly converted by PCR into a template for in vitro mRNA synthesis using appropriate primers and RNA polymerase. In one embodiment, the desired template for in vitro transcription is human TEM1, or TEM1-TT or a combination thereof.

In one embodiment, the oligonucleotide to be used for PCR contains an open reading frame. In one embodiment, the oligonucleotide is a full length TEM1 gene or a portion of TEM1 gene. The gene can include some or all of the 5′ and/or 3′ untranslated regions (UTRs). The gene can include exons and introns. In one embodiment, the oligonucleotide to be used for PCR is a human TEM1 gene. In another embodiment, the oligonucleotide to be used for PCR is a human TEM1 gene including the 5′ and 3′ UTRs. The oligonucleotide can alternatively be an artificial TEM1 sequence that is not normally expressed in a naturally occurring organism. An exemplary artificial oligonucleotide TEM1 sequence is one that contains portions of TEM1 gene that is ligated together to form an open reading frame that encodes a fusion protein, e.g., tetanus toxoid.

In various embodiments, a plasmid is used to generate a template for in vitro transcription of mRNA, which is used as the active vaccine component.

Chemical structures with the ability to promote stability and/or translation efficiency may also be used. In certain embodiments, the oligonucleotide encoding the mRNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency.

The 5′ and 3′ UTRs can be the naturally occurring, endogenous 5′ and 3′ UTRs for TEM1 gene. Alternatively, UTR sequences that are not endogenous to TEM1 gene can be added by incorporating the UTR sequences into the forward and reverse primers or by any other modifications of the template. The use of UTR sequences that are not endogenous to the gene of interest can be useful for modifying the stability and/or translation efficiency of the mRNA. For example, it is known that AU-rich elements in 3′ UTR sequences can decrease the stability of mRNA. Therefore, 3′ UTRs can be selected or designed to increase the stability of the transcribed mRNA based on properties of UTRs that are well known in the art.

In one embodiment, the 5′ UTR can contain the Kozak sequence of TEM1 gene. Alternatively, when a 5′ UTR that is not endogenous to TEM1 gene of interest is being added by PCR as described above, a consensus Kozak sequence can be redesigned by adding the 5′ UTR sequence. Kozak sequences can increase the efficiency of translation of some mRNA transcripts, but does not appear to be required for all mRNAs to enable efficient translation. The requirement for Kozak sequences for many mRNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the mRNA.

To enable synthesis of the mRNA from a DNA template without the need for gene cloning, a promoter of transcription should or can be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the mRNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the mRNA.

5′ caps on also provide stability to mRNA molecules. In one embodiment, RNAs produced by the methods to include a 5′ cap1 structure. Such cap1 structure can be generated using Vaccinia capping enzyme and 2′-O-methyltransferase enzymes (CellScript, Madison, Wis.). Alternatively, 5′ cap is provided using techniques known in the art and described in, for example, Cougot, et al., Trends in Biochem. Sci., 2001, 29, pp. 436-444; Stepinski, et al., RNA, 2001, 7, pp. 1468-95; and Elango, et al., Biochim. Biophys. Res. Commun., 2005, 330, pp. 958-966.

mRNA can be introduced into the subject using any of a number of different methods, for instance, using protamine (cationic peptide)-complexed mRNA; mRNA associated with a positively charged oil-in-water cationic nanoemulsion; mRNA associated with a chemically modified dendrimer and complexed with polyethylene glycol (PEG)-lipid; protamine-complexed mRNA in a PEG-lipid nanoparticle; mRNA associated with a cationic polymer such as polyethylenimine (PEI); mRNA associated with a cationic polymer such as PEI and a lipid component; mRNA associated with a polysaccharide (e.g., chitosan); mRNA in a cationic lipid nanoparticle (e.g., 1,2-dioleoyloxy-3-trimethylammoniumpropane (DOTAP) or dioleoylphosphatidylethanolamine (DOPE) lipids); mRNA complexed with cationic lipids and cholesterol; mRNA complexed with cationic lipids, cholesterol, and PEG-lipid; or mRNA complexed with cationic lipids, neutral lipids, cholesterol, and PEG-lipid.

In one embodiment, the composition of the present invention comprises a nucleoside-modified nucleic acid encoding the mRNA as described herein. For example, in one embodiment, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. Nos. 8,278,036, 8,691,966, and 8,835,108, all of which are incorporated by reference herein in their entirety.

In certain embodiments, nucleoside-modified mRNA does not activate any pathophysiologic pathways, translates very efficiently and almost immediately following delivery, and serve as templates for continuous protein production in vivo lasting for several days. See, for example, Kariko et al., Mol Ther., 2008, 16, pp. 1833-1840 and Kariko et al., Mol Ther., 2012, 20, pp. 948-953. The amount of mRNA required to exert a physiological effect is small and that makes it applicable for human therapy. For example, as described herein, nucleoside-modified mRNA has demonstrated the ability to antigen-specific antibody production. For example, in certain instances, antigen encoded by nucleoside-modified mRNA induces greater production of antigen-specific antibody production as compared to antigen encoded by non-modified mRNA.

In certain instances, expressing a protein by delivering the encoding mRNA has many benefits over methods that use protein or plasmid DNA. During mRNA transfection, the coding sequence of the desired protein is the only substance delivered to cells, thus avoiding all the side effects associated with plasmid backbones. More importantly, unlike DNA-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects.

In certain embodiments, the nucleoside-modified mRNA comprises the naturally occurring modified-nucleoside pseudouridine. In certain embodiments, inclusion of pseudouridine makes the mRNA more stable, non-immunogenic, and highly translatable. See, for example, Kariko et al., Mol Ther., 2008, 16, pp. 1833-1840; Anderson et al., Nucleic Acids Res., 2010, 38, pp. 5884-5892; Anderson et al., Nucleic Acids Research, 2011, 39, pp. 9329-9338; Kariko et al., Nucleic Acids Research, 2011, 39, e142; Kariko et al., Mol Ther., 2012, 20, pp. 948-953; and Kariko et al., Immunity, 2005, 23, pp. 165-175.

It has been demonstrated that the presence of modified nucleosides, including pseudouridines in mRNA suppress their innate immunogenicity. Further, protein-encoding, in vitro-transcribed mRNA containing pseudouridine can be translated more efficiently than mRNA containing no or other modified nucleosides. Subsequently, it is shown that the presence of pseudouridine improves the stability of mRNA and abates both activation of PKR and inhibition of translation.

In certain embodiments, the nucleoside-modified nucleic acid molecule is a purified nucleoside-modified nucleic acid molecule. For example, in certain embodiments, the composition is purified to remove double-stranded contaminants. In certain instances, a preparative high performance liquid chromatography (HPLC) purification procedure is used to obtain pseudouridine-containing mRNA that has superior translational potential and no innate immunogenicity. Administering HPLC-purified, pseudourine-containing mRNA coding for erythropoietin into mice and macaques resulted in a significant increase of serum EPO levels, thus confirming that pseudouridine-containing mRNA is suitable for in vivo protein therapy. In certain embodiments, the nucleoside-modified nucleic acid molecule is purified using non-HPLC methods. In certain instances, the nucleoside-modified nucleic acid molecule is purified using chromatography methods, including but not limited to HPLC and fast protein liquid chromatography (FPLC). An exemplary FPLC-based purification procedure is described in Weissman et al., Methods Mol Biol., 2013, 969, pp. 43-54. Exemplary purification procedures are also described in U.S. Patent Application Publication No. US2016/0032316, which is hereby incorporated by reference in its entirety.

The present invention encompasses mRNA comprising pseudouridine or a modified nucleoside. In certain embodiments, the composition comprises an isolated mRNA, wherein the mRNA comprises a pseudouridine or a modified nucleoside. In certain embodiments, the composition comprises a vector, comprising an isolated oligonucleotide that encodes the mRNA of the invention, wherein the oligonucleotide encodes the mRNA of the invention that comprises a pseudouridine or a modified nucleoside.

In one embodiment, the nucleoside-modified mRNA of the invention is IVT RNA. For example, in certain embodiments, the nucleoside-modified mRNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine). In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA (i.e., mRNA) of the present invention is uridine (U). In another embodiment, the modified nucleoside is cytidine (C). In another embodiment, the modified nucleoside is adenosine (A). In another embodiment, the modified nucleoside is guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is T (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m6A (2-methylthio-N⁶-methyladenosine); i⁶A (N⁶-isopentenyladenosine); ms²i6A (2-methylthio-N⁶-isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t6A (2-methylthio-N⁶-threonyl carbamoyladenosine); m⁶t6A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); miI (1-methylinosine); miIm (1,2′-O-dimethylinosine); m³C (3-methylcytidine); Cm (2′-O-methylcytidine); s²C (2-thiocytidine); ac⁴C (N⁴-acetylcytidine); fVC (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m?G (7-methylguanosine); Gm (2′-O-methylguanosine); m²2G (N²,N²-dimethylguanosine); m²Gm (N²,2′-O-dimethylguanosine); m²2Gm (N.sup.2,N.sup.2,2′-O-trimethylguanosine); Gr(p) (2′-O-ribosylguanosine (phosphate)); yW (wybutosine); O₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQo (7-cyano-7-deazaguanosine); preQi (7-aminomethyl-7-deazaguanosine); G+(archaeosine); D (dihydrouridine); m⁵Um (5,2′-O-dimethyluridine); s⁴U (4-thiouridine); m.⁵s2U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm²Um (5-methoxycarbonylmethyl-2′-O-methyluridine); mcm²s2U (5-methoxycarbonylmethyl-2-thiouridine); nm⁵s2U (5-aminomethyl-2-thiouridine); mnm⁵U (5-methylaminomethyluridine); mnm⁵s2U (5-methylaminomethyl-2-thiouridine); mnm⁵se²U (5-methylaminomethyl-2-selenouridine); nCm⁵U (5-carbamoylmethyluridine); nCm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s2U (5-carboxymethylaminomethyl-2-thiouridine); m⁶2A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶2Am (N⁶,N⁶,0-2′-trimethyladenosine); m²,7G (N²,7-dimethylguanosine); m²,2,⁷G (N²,N²,7-trimethylguanosine); m³Um (3,2′-O-dimethyluridine); m⁵D (5-methyldihydrouridine); f⁵Cm (5-formyl-2′-O-methylcytidine); miGm (1,2′-O-dimethylguanosine); miAm (1,2′-O-dimethyladenosine); im⁵U (5-taurinomethyluridine); im⁵s2U (5-taurinomethyl-2-thiouridine)); imG-14 (4-demethylwyosine); imG2 (isowyosine); or ac⁶A (N.sup.6-acetyladenosine).

In another embodiment, a nucleoside-modified mRNA of the present invention comprises a combination of 2 or more of the above modifications. In another embodiment, the nucleoside-modified mRNA comprises a combination of 3 or more of the above modifications. In another embodiment, the nucleoside-modified mRNA comprises a combination of more than 3 of the above modifications.

In various embodiments, between 0.1% and 100% of the residues in the nucleoside-modified mRNA of the present invention are modified (e.g., either by the presence of pseudouridine or another modified nucleoside base). In one embodiment, the fraction of modified residues is 0.1%. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%. In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified mRNA of the present invention is translated in the cell more efficiently than an unmodified mRNA molecule with the same sequence. In another embodiment, the nucleoside-modified mRNA exhibits enhanced ability to be translated by a subject. In another embodiment, translation is enhanced by a factor of 2-fold relative to its unmodified counterpart. In another embodiment, translation is enhanced by a 3-fold factor. In another embodiment, translation is enhanced by a 4-fold factor. In another embodiment, translation is enhanced by a 5-fold factor. In another embodiment, translation is enhanced by a 6-fold factor. In another embodiment, translation is enhanced by a 7-fold factor. In another embodiment, translation is enhanced by an 8-fold factor. In another embodiment, translation is enhanced by a 9-fold factor. In another embodiment, translation is enhanced by a 10-fold or more factor.

In another embodiment, the nucleoside-modified mRNA of the present invention provide improved stability, thereby increasing the T_{1/2} (i.e., half-life) as compared with an unmodified in vitro-synthesized mRNA molecule of the same sequence. In another embodiment, the modified RNA molecule provide at least 2-fold greater half-life than its unmodified counterpart. In another embodiment, the half-life is increased by at least a 3-fold factor. In another embodiment the half-life is increased by at least a 5-fold factor.

In another embodiment, the modified mRNA can induce significantly more robust adaptive immune response. The phrase “induces significantly more robust adaptive immune response” refers to a detectable increase in an adaptive immune response. In another embodiment, the term refers to a fold increase in the adaptive immune response (e.g., at least 2-fold, 3-fold, or 4-fold increase in immune response). In another embodiment, the term refers to an increase such that the nucleoside-modified mRNA can be administered at a lower dose or frequency than an unmodified mRNA molecule while still inducing a similarly effective adaptive immune response. In another embodiment, the increase is such that the nucleoside-modified mRNA can be administered using a single dose to induce an effective adaptive immune response.

In another embodiment, the nucleoside-modified mRNA of the present invention exhibits significantly less innate immunogenicity than an unmodified in vitro-synthesized mRNA molecule of the same sequence. In another embodiment, the modified mRNA molecule exhibits an innate immune response that is 2-fold less than its unmodified counterpart. In another embodiment, innate immunogenicity is reduced by a 3-fold factor. In another embodiment, innate immunogenicity is reduced by a 4-fold factor. In another embodiment, innate immunogenicity is reduced by a 5-fold factor or more.

In another embodiment, “exhibits significantly less innate immunogenicity” refers to a detectable decrease in innate immunogenicity. In another embodiment, the term refers to a fold decrease in innate immunogenicity. In another embodiment, the term refers to a decrease such that an effective amount of the nucleoside-modified mRNA can be administered without triggering a detectable innate immune response. In another embodiment, the term refers to a decrease such that the nucleoside-modified mRNA can be repeatedly administered without eliciting an innate immune response.

In one embodiment, delivery of mRNA of the invention comprises any suitable delivery method, including exemplary mRNA transfection methods known to one skilled in the art. In certain embodiments, delivery of mRNA of the invention to a subject comprises mixing the mRNA with a transfection reagent prior to the step of contacting. In another embodiment, a method of present invention further comprises administering mRNA together with the transfection reagent. In another embodiment, the transfection reagent is a cationic lipid reagent. In another embodiment, the transfection reagent is a cationic polymer reagent.

In another embodiment, the transfection reagent is a lipid-based transfection reagent. In another embodiment, the transfection reagent is a protein-based transfection reagent. In another embodiment, the transfection reagent is a carbohydrate-based transfection reagent. In another embodiment, the transfection reagent is a cationic lipid-based transfection reagent. In another embodiment, the transfection reagent is a cationic polymer-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids, which make up the cell membrane. They have, in another embodiment, an internal aqueous space for entrapping water-soluble compounds and range in size from 0.05 to several microns in diameter. In another embodiment, liposomes can deliver mRNA to cells in a biologically active form.

In one embodiment, the composition comprises a lipid nanoparticle (LNP) and one or more mRNA of the invention. For example, in one embodiment, the composition comprises an LNP and TEM1 mRNA, TEM1 mRNA-TT mRNA, or a combination thereof.

The term “lipid nanoparticle” refers to a particle having at least one dimension on the order of nanometers (e.g., 1-1,000 nm). Suitable lipid nanoparticles are well known to one skilled in the art. In some embodiments, lipid nanoparticles are included in a formulation comprising an mRNA of the invention. In some embodiments, such lipid nanoparticles comprise a cationic lipid and one or more excipient selected from neutral lipids, charged lipids, steroids and polymer conjugated lipids (e.g., a pegylated lipid). In some embodiments, the mRNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the mRNA of the invention, when present in the lipid nanoparticles, is resistant in aqueous solution to degradation with a nuclease.

The LNP may comprise any lipid capable of forming a particle to which the one or more mRNA molecules are attached, or in which the one or more mRNA molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethyl-ammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethyl-ammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, which is incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers. In certain embodiments, the formulation may comprise dry particles which comprise the active ingredient and which have a diameter in the range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In certain embodiments, such powders comprise particles wherein at least 98% of the particles by weight have a diameter greater than 0.5 nanometers and at least 95% of the particles by number have a diameter less than 7 nanometers. In certain embodiments, at least 95% of the particles by weight have a diameter greater than 1 nanometer and at least 90% of the particles by number have a diameter less than 6 nanometers. In certain embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in certain instances having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Below steps show some of the key processes for producing a representative TEM1 mRNA vaccine of the invention:
Design TEM1 mRNA constructs
Design and develop plasmid DNA with TEM1 mRNA constructs
Tem1-TT mRNA (Tumor Endothelial Marker 1 fused with Tetanus Toxoid)
In-vitro transcription and purification of TEM1 mRNA
The mRNA sequence may have modified nucleosides, such as pseudouridine, N1-methylpseudouridine or other modified nucleoside analogues.
Conduct validation studies for TEM1 protein expression in mammalian cell
Conduct stability studies of TEM1 mRNA vaccine
LNP formulation for TEM1 mRNA vaccine delivery

Below is a list of some of the methods for delivering mRNA of the invention to a subject:
protamine (cationic peptide)-complexed mRNA
mRNA associated with a positively charged oil-in-water cationic nanoemulsion
mRNA associated with a chemically modified dendrimer and complexed with polyethylene glycol (PEG)-lipid
protamine-complexed mRNA in a PEG-lipid nanoparticle
mRNA associated with a cationic polymer such as polyethylenimine (PEI)
mRNA associated with a cationic polymer such as PEI and a lipid component
mRNA associated with a polysaccharide (e.g., chitosan)
mRNA in a cationic lipid nanoparticle (e.g., 1,2-dioleoyloxy-3-trimethylammoniumpropane (DOTAP) or dioleoylphosphatidylethanolamine (DOPE) lipids)
mRNA complexed with 1,2-dioleyloxy-3-dimethylaminopropane (DODMA), 18:1 (Δ9-Cis) PE (DOPE), cholesterol, and DMG-PEG 2000 (DMG PEG)
mRNA complexed with cationic lipids and cholesterol
mRNA complexed with cationic lipids, cholesterol, and PEG-lipid
mRNA complexed with cationic lipids, neutral lipids, cholesterol, and PEG-lipid

Transient Plasmid DNA Transfection: In a 6 well plate, 200,000 BEK 293 cells were placed along with the growth media, Dulbecco's Modified Eagle's Medium (DMEM) supplemented with 10% Fetal Bovine Serum (FBS) and 1% Penicillin/Streptomycin antibiotics. The growth media was removed and Opti-MEM media was added before transfection. Briefly, transfection was carried out as follows:

Lipofectamine 3000 transfection reagent (Invitrogen, Cat No: L3000-008):
Solution A: 125 μL/well Opti MEM+6 μL/well Lipofectamine 3000 reagent
Solution B: 125 μL/well Opti MEM+TEM 1/TEM1-TT Plasmid DNA 1.5 μg/μl+P3000 Regent 2 μL/μg of plasmid DNA
   On day 1, solutions A and B were mixed and incubated for 15 min. The incubated solution was added into the cells. After 6 h, media was replaced with complete media (Dulbecco's Modified Eagle's Medium (DMEM) supplemented with 10% Fetal Bovine Serum (FBS)) after five hours. On days 3 and 4, the cells were harvested at desired timepoint (48 h) to make cell lysate for western blot/RNA isolation.

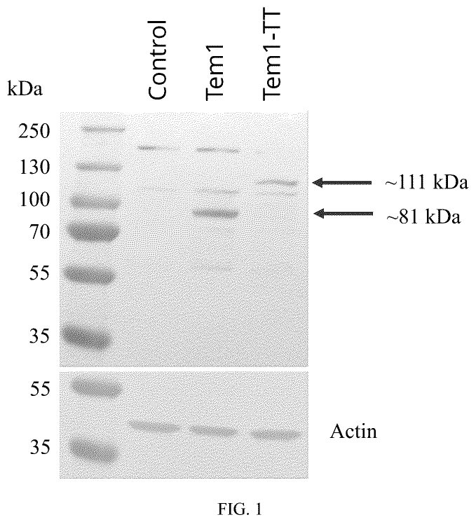

Tem1 and Tem1-TT plasmid DNA were transfected using Lipofectamine-3000 transfection reagent. Protein expression was analyzed with western blotting using Tem1 polyclonal antibody. Detection of transient expression of Tem1 protein with Tem1 and Tem1-TT plasmid DNA confirmed the specificity of the plasmid for targeting Tem1 protein. See FIG. 1.

RNA Expression Analysis from Plasmid DNA Transfection: Tem1 and Tem1-TT plasmid DNA were transfected using Lipofectamine-3000 transfection reagent as discussed above. Cells were harvested and RNA isolation was performed with PureLink RNA Mini Kit (Invitrogen, Cat No: 12183018A). cDNA was synthesized using High-Capacity cDNA Reverse Transcription Kit (Applied Biosystems, Cat No. 4374966). 200 ng of RNA was converted to cDNA using High-Capacity cDNA Reverse Transcription Kit (Applied Biosystems, Cat No. 4374966) containing random hexamer primers, and PCR reactions were assembled in PCR tubes using 1 μl of a cDNA reaction, 400 nM of target specific primer mix under the following conditions:

| Step | Cycles | Temperature | | Time | |
|---|---|---|---|---|---|
| Activation | 1 | 94° | C. | 2 | min |
| Denaturation | 40 | 94° | C. | 15 | sec |
| Annealing | | 60° | C. | 15 | sec |
| Extension | | 68° | C. | 20 | sec/kb |

| | | | | |
|---|---|---|---|---|
| Hold | 1 | 4° | C. | Hold |

| | | Amplicon |
|---|---|---|
| Primer | Sequence | Size |
| Primer 1 (P1) | F: CCTGGAATCACATCAGCTACTC (Sense | 621 |
| (TEM 1) | (SEQ ID NO: 26) | |
| | R: ACGGGTGCAGTACACAATAC (Anti-Sense) | |
| | (SEQ ID NO: 27) | |
| Primer 2 (P2) | F: GACCAGGATACCGCCTTTAC (Sense) | 427 |
| (TEM 1) | (SEQ ID NO: 28) | |
| | R: CCACCTCTTCCACACATTCA (Anti-Sense) | |
| | (SEQ ID NO: 29) | |
| Primer 3 (P3) | F: CTCAGTTGGTGCCAGGTATAA (Sense) | 494 |
| (TEM1-TT) | (SEQ ID NO: 30) | |
| | R: CCTATCCAGCTTGAGGGTAATG (Anti-Sense) | |
| | (SEQ ID NO: 31) | |
| Primer 5 (P5) | F: CCTGTGTCAATTTGGGTTTGAG (Sense) | 796 |
| (TEM1-TT) | (SEQ ID NO: 32) | |
| | R: CTCTCCATCCTGTGGGAAATTAG (Anti-Sense) | |
| | (SEQ ID NO: 33) | |

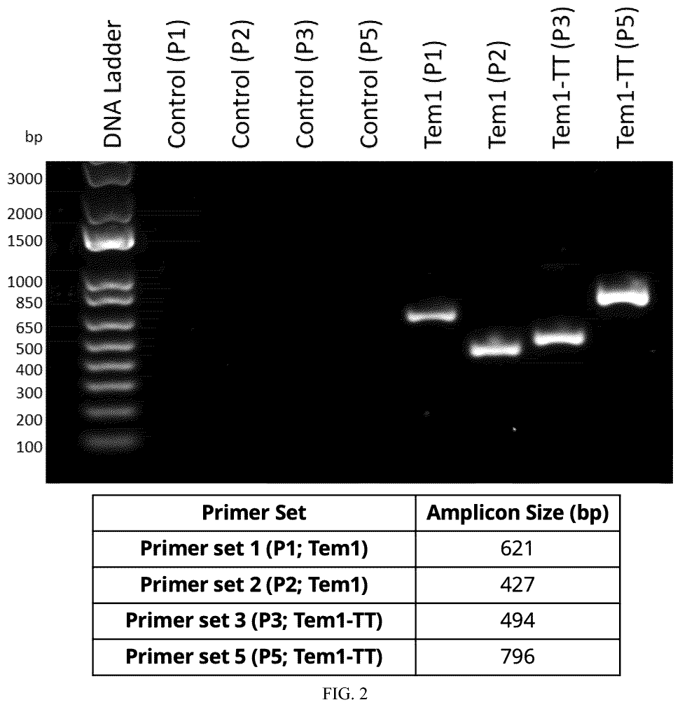

PCR result with the respective amplicon size confirmed the plasmid DNA construct design for Tem1 and Tem1-TT. See FIG. 2.

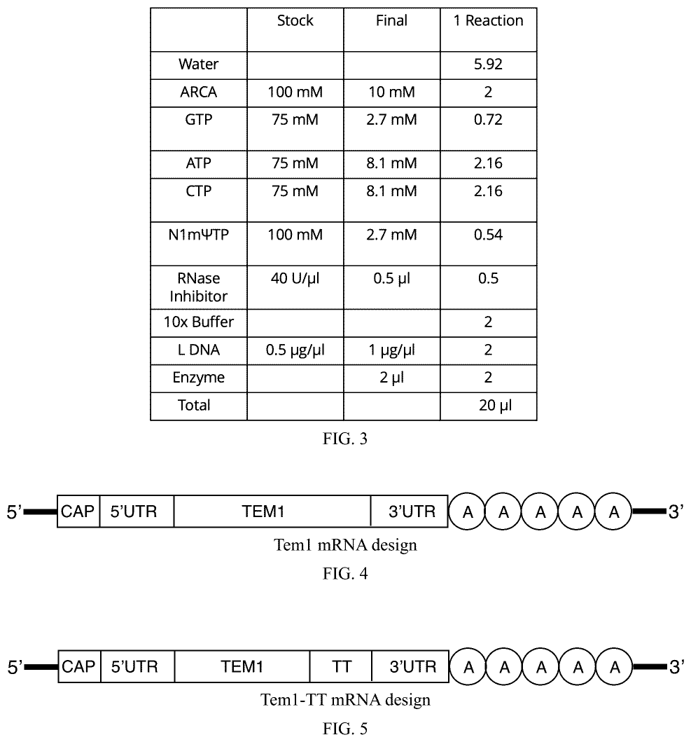

Synthesis and Purification of mRNA: Template directed synthesis of RNA molecule was done by In vitro Transcription using 5×MEGAscript T7 Kit (Invitrogen, Cat No: AMB1334-5). The volume of components needed was calculated to prepare the required number of reactions and added into the microfuge tube. FIG. 3. The reaction mixture was incubated at 37° C. for 4 hr in a thermomixer. To remove the template DNA, 1 μl of DNase was added per reaction mixture and incubated 15 min at 37° C. 5 μl of 10× Antarctic phosphatase buffer and 3 μl of Antarctic phosphatase were added to each reaction. The reaction mixture was incubated at 37° C. for 15 min in a thermomixer and heat deactivation of enzyme was done by heating the reaction mixture at 80° C. Each reaction was purified using MEGAclear Kit (Invitrogen, Cat No: AM1908) using RNA elution option 1.

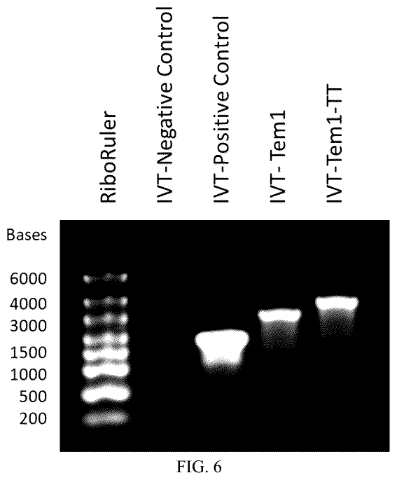

In-Vitro Transcription (IVT): In-Vitro mRNA Synthesis: mRNA was synthesized using T7 in-vitro mRNA synthesis kit from a linearized plasmid DNA template (FIG. 4). mRNA was synthesized with N1-methyl pseudouridine (m1Ψ) and a cap structure, anti-reverse cap analogs (ARCA) (FIG. 5). Integrity and quality of mRNA were confirmed with agarose gel electrophoresis. FIG. 6.

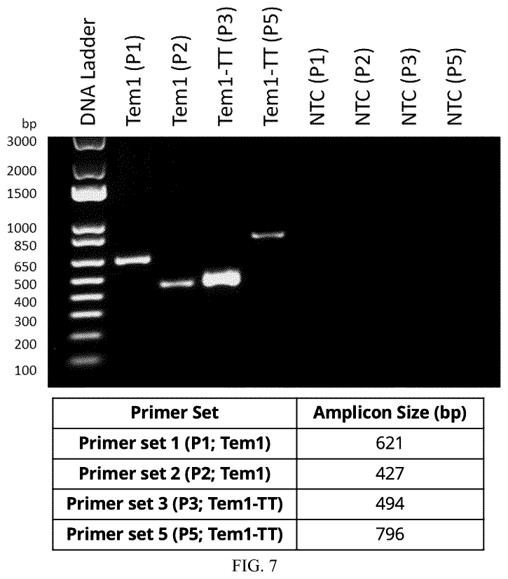

Characterization of In-Vitro Synthesized mRNA: TEM1 and TEM1-TT mRNA was synthesized using T7 in-vitro transcription kit. cDNA was synthesized from mRNA and used for RNA expression analysis using specific primer sets. PCR result with the respective amplicon size confirms the integrity and specificity of in vitro synthesized Tem1 and Tem1-TT mRNA. FIG. 7.

Plasmid construct design for Circular RNA based on T4 RNA Ligase method: Circular RNA lacks the essential elements for cap dependent translation, it can be engineered to enable protein translation through an internal ribosome entry site (IRES) upstream of the open reading frame (ORF). A circular RNA construct for TEM1 (SEQ ID NO: 24) and TEM1-TT (SEQ ID NO: 25) is designed and tested.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

Sequence Listing:

Sequence Number (ID): 1
Length: 2274
Molecule Type: DNA
Features Location/Qualifiers:
- source, 1 . . . 2274
> mol type, genomic DNA
> organism, Homo sapiens
Residues:
atgctgctgc gcctgttgct ggcctgggcg gccgcagggc ccacactggg ccaggacccc 60
tgggctgctg agccccgtgc cgcctgcggc cccagcagct gctacgctct cttcccacgg 120
cgccgcacct tcctggaggc ctggcgggcc tgccgcgagc tggggggcga cctggccact 180
cctcggaccc ccgaggaggc ccagcgtgtg gacagcctgg tgggtgcggg cccagccagc 240
cggctgctgt ggatcgggct gcagcggcag gcccggcaat gccagctgca gcgcccactg 300
cgcggcttca cgtggaccac aggggaccag gacacggctt tcaccaactg ggcccagcca 360
gcctctggag gcccctgccc ggcccagcgc tgtgtggccc tggaggcaag tggcgagcac 420
cgctggctgg agggctcgtg cacgctggct gtcgacggct acctgtgcca gtttggcttc 480
gagggcgcct gcccggcgct gcaagatgag gcgggccagg ccggcccagc cgtgtatacc 540
acgcccttcc acctggtctc cacagagttt gagtggctgc ccttcggctc tgtggccgct 600
gtgcagtgcc aggctggcag gggagcctct ctgctctgcg tgaagcagcc tgagggaggt 660
gtgggctggt cacgggctgg gcccctgtgc ctggggactg gctgcagccc tgacaacggg 720
ggctgcgaac acgaatgtgt ggaggaggtg gatggtcacg tgtcctgccg ctgcactgag 780
ggcttccggc tggcagcaga cgggcgcagt tgcgaggacc cctgtgccca ggctccgtgc 840
gagcagcagt gtgagcccgg tgggccacaa ggctacagct gccactgtcg cctgggtttc 900
cggccagcgg aggatgatcc gcaccgctgt gtggacacag atgagtgcca gattgccggt 960
gtgtgccagc agatgtgtgt caactacgtt ggtggcttcg agtgttattg tagcgaggga 1020
catgagctgg aggctgatgg catcagctgc agccctgcag gggccatggg tgcccaggct 1080
tcccaggacc tcggagatga gttgctggat gacggggagg atgaggaaga tgaagacgag 1140
gcctggaagg ccttcaacgg tggctggacg gagatgcctg ggatcctgtg gatggagcct 1200
acgcagccgc ctgactttgc cctggcctat agaccgagct tcccagagga cagagagcca 1260
cagataccct acccggagcc cacctggcca cccccgctca gtgcccccag ggtcccctac 1320
cactcctcag tgctctccgt cacccggcct gtggtggtct ctgccacgca tcccacactg 1380
ccttctgccc accagcctcc tgtgatccct gccacacacc cagctttgtc ccgtgaccac 1440
cagatccccg tgatcgcagc caactatcca gatctgcctt ctgcctacca acccggtatt 1500
ctctctgtct ctcattcagc acagcctcct gcccaccagc cccctatgat ctcaaccaaa 1560
tatccggagc tcttccctgc ccaccagtcc cccatgtttc cagacacccg ggtcgctggc 1620
acccagacca ccactcattt gcctggaatc ccacctaacc atgcccctct ggtcaccacc 1680
ctcggtgccc agctaccccc tcaagcccca gatgcccttg tcctcagaac ccaggccacc 1740
cagcttccca ttatcccaac tgcccagccc tctctgacca ccacctccag gtcccctgtg 1800
tctcctgccc atcaaatctc tgtgcctgct gccacccagc ccgcagccct ccccaccctc 1860
ctgccctctc agagccccac taaccagacc tcacccatca gccctacaca tccccattcc 1920
aaagcccccc aaatcccaag ggaagatggc cccagtccca agttggccct gtggctgccc 1980
tcaccagctc ccacagcagc cccaacagcc ctgggggagg ctggtcttgc cgagcacagc 2040
cagagggatg accggtggct gctggtggca ctcctggtgc caacgtgtgt ctttttggtg 2100
gtcctgcttg cactgggcat cgtgtactgc acccgctgtg gcccccatgc acccaacaag 2160
cgcatcactg actgctatcg ctgggtcatc catgctggga gcaagagccc aacagaaccc 2220
atgcccccca ggggcagcct cacaggggtg cagacctgca gaaccagcgt gtga 2274
Sequence Number (ID): 2
Length: 2274
Molecule Type: RNA
Features Location/Qualifiers:
- source, 1 . . . 2274
> mol type, mRNA
> organism, Homo sapiens
Residues:
atgctgctgc gcctgttgct ggcctgggcg gccgcagggc ccacactggg ccaggacccc 60
tgggctgctg agccccgtgc cgcctgcggc cccagcagct gctacgctct cttcccacgg 120
cgccgcacct tcctggaggc ctggcgggcc tgccgcgagc tggggggcga cctggccact 180
cctcggaccc ccgaggaggc ccagcgtgtg gacagcctgg tgggtgcggg cccagccagc 240
cggctgctgt ggatcgggct gcagcggcag gcccggcaat gccagctgca gcgcccactg 300
cgcggcttca cgtggaccac aggggaccag gacacggctt tcaccaactg ggcccagcca 360
gcctctggag gcccctgccc ggcccagcgc tgtgtggccc tggaggcaag tggcgagcac 420
cgctggctgg agggctcgtg cacgctggct gtcgacggct acctgtgcca gtttggcttc 480
gagggcgcct gcccggcgct gcaagatgag gcgggccagg ccggcccagc cgtgtatacc 540
acgcccttcc acctggtctc cacagagttt gagtggctgc ccttcggctc tgtggccgct 600
gtgcagtgcc aggctggcag gggagcctct ctgctctgcg tgaagcagcc tgagggaggt 660
gtgggctggt cacgggctgg gcccctgtgc ctggggactg gctgcagccc tgacaacggg 720
ggctgcgaac acgaatgtgt ggaggaggtg gatggtcacg tgtcctgccg ctgcactgag 780
ggcttccggc tggcagcaga cgggcgcagt tgcgaggacc cctgtgccca ggctccgtgc 840
gagcagcagt gtgagcccgg tgggccacaa ggctacagct gccactgtcg cctgggtttc 900
cggccagcgg aggatgatcc gcaccgctgt gtggacacag atgagtgcca gattgccggt 960
gtgtgccagc agatgtgtgt caactacgtt ggtggcttcg agtgttattg tagcgaggga 1020
catgagctgg aggctgatgg catcagctgc agccctgcag gggccatggg tgcccaggct 1080
tcccaggacc tcggagatga gttgctggat gacggggagg atgaggaaga tgaagacgag 1140
gcctggaagg ccttcaacgg tggctggacg gagatgcctg ggatcctgtg gatggagcct 1200
acgcagccgc ctgactttgc cctggcctat agaccgagct tcccagagga cagagagcca 1260
cagataccct acccggagcc cacctggcca cccccgctca gtgcccccag ggtcccctac 1320
cactcctcag tgctctccgt cacccggcct gtggtggtct ctgccacgca tcccacactg 1380
ccttctgccc accagcctcc tgtgatccct gccacacacc cagctttgtc ccgtgaccac 1440
cagatccccg tgatcgcagc caactatcca gatctgcctt ctgcctacca acccggtatt 1500
ctctctgtct ctcattcagc acagcctect gcccaccagc cccctatgat ctcaaccaaa 1560
tatccggagc tettccctgc ccaccagtcc cccatgtttc cagacacccg ggtcgctggc 1620
acccagacca ccactcattt gcctggaatc ccacctaacc atgcccctct ggtcaccacc 1680
ctcggtgccc agctaccccc tcaagcccca gatgcccttg tcctcagaac ccaggccacc 1740
cagcttccca ttatcccaac tgcccagccc tctctgacca ccacctccag gtcccctgtg 1800
tctcctgccc atcaaatctc tgtgcctgct gccacccagc ccgcagccct ccccaccctc 1860
ctgccctctc agagccccac taaccagacc tcacccatca gccctacaca tccccattcc 1920
aaagcccccc aaatcccaag ggaagatggc cccagtccca agttggccct gtggctgccc 1980
tcaccagctc ccacagcagc cccaacagcc ctgggggagg ctggtcttgc cgagcacagc 2040
cagagggatg accggtggct gctggtggca ctcctggtgc caacgtgtgt ctttttggtg 2100
gtcctgcttg cactgggcat cgtgtactgc acccgctgtg gcccccatgc acccaacaag 2160
cgcatcactg actgctatcg ctgggtcatc catgctggga gcaagagccc aacagaaccc 2220
atgcccccca ggggcagcct cacaggggtg cagacctgca gaaccagcgt gtga 2274
Sequence Number (ID): 3
Length: 757
Molecule Type: AA
Features Location/Qualifiers:
- source, 1 . . . 757
> mol type, protein
> organism, Homo sapiens
Residues:
MLLRLLLAWA AAGPTLGQDP WAAEPRAACG PSSCYALFPR RRTFLEAWRA CRELGGDLAT 60
PRTPEEAQRV DSLVGAGPAS RLLWIGLORO ARQCOLORPL RGFTWTTGDQ DTAFTNWAQP 120
ASGGPCPAQR CVALEASGEH RWLEGSCTLA VDGYLCQFGF EGACPALQDE AGQAGPAVYT 180
TPFHLVSTEF EWLPEGSVAA VQCQAGRGAS LLCVKQPEGG VGWSRAGPLC LGTGCSPDNG 240
GCEHECVEEV DGHVSCRCTE GFRLAADGRS CEDPCAQAPC EQQCEPGGPQ GYSCHCRLGF 300
RPAEDDPHRC VDTDECQIAG VCQQMCVNYV GGFECYCSEG HELEADGISC SPAGAMGAQA 360
SQDLGDELLD DGEDEEDEDE AWKAFNGGWT EMPGILWMEP TOPPDFALAY RPSFPEDREP 420
QIPYPEPTWP PPLSAPRVPY HSSVLSVTRP VVVSATHPTL PSAHOPPVIP ATHPALSRDH 480
QIPVIAANYP DLPSAYQPGI LSVSHSAQPP AHQPPMISTK YPELFPAHQS PMFPDTRVAG 540
TOTTTHLPGI PPNHAPLVTT LGAQLPPQAP DALVLRTQAT QLPIIPTAQP SLTTTSRSPV 600
SPAHQISVPA ATQPAALPTL LPSQSPTNQT SPISPTHPHS KAPQIPREDG PSPKLALWLP 660
SPAPTAAPTA LGEAGLAEHS QRDDRWLLVA LLVPTCVELV VLLALGIVYC TRCGPHAPNK 720
RITDCYRWVI HAGSKSPTEP MPPRGSLTGV QTCRTSV 757
Sequence Number (ID): 4
Length: 3072
Molecule Type: DNA
Features Location/Qualifiers:
- source, 1 . . . 3072
> mol type, other DNA
> organism, synthetic construct
- gene, 1 . . . 2274
> gene, Human TEM1 DNA
- misc feature, 2275 . . . 2304
> note, Linker
- gene, 2305 . . . 3072
> gene, Tetanus Toxoid
Residues:
atgctgctgc gcctgttgct ggcctgggcg gccgcagggc ccacactggg ccaggacccc 60
tgggctgctg agccccgtgc cgcctgcggc cccagcagct gctacgctct cttcccacgg 120
cgccgcacct tcctggaggc ctggcgggcc tgccgcgagc tggggggcga cctggccact 180
cctcggaccc ccgaggaggc ccagcgtgtg gacagcctgg tgggtgcggg cccagccagc 240
cggctgctgt ggatcgggct gcagcggcag gcccggcaat gccagctgca gcgcccactg 300
cgcggcttca cgtggaccac aggggaccag gacacggctt tcaccaactg ggcccagcca 360
gcctctggag gcccctgccc ggcccagcgc tgtgtggccc tggaggcaag tggcgagcac 420
cgctggctgg agggctcgtg cacgctggct gtcgacggct acctgtgcca gtttggcttc 480
gagggcgcct gcccggcgct gcaagatgag gcgggccagg ccggcccagc cgtgtatacc 540
acgcccttcc acctggtctc cacagagttt gagtggctgc ccttcggctc tgtggccgct 600
gtgcagtgcc aggctggcag gggagcctct ctgctctgcg tgaagcagcc tgagggaggt 660
gtgggctggt cacgggctgg gcccctgtgc ctggggactg gctgcagccc tgacaacggg 720
ggctgcgaac acgaatgtgt ggaggaggtg gatggtcacg tgtcctgccg ctgcactgag 780
ggcttccggc tggcagcaga cgggcgcagt tgcgaggacc cctgtgccca ggctccgtgc 840
gagcagcagt gtgagcccgg tgggccacaa ggctacagct gccactgtcg cctgggtttc 900
cggccagcgg aggatgatcc gcaccgctgt gtggacacag atgagtgcca gattgccggt 960
gtgtgccagc agatgtgtgt caactacgtt ggtggcttcg agtgttattg tagcgaggga 1020
catgagctgg aggctgatgg catcagctgc agccctgcag gggccatggg tgcccaggct 1080
tcccaggacc tcggagatga gttgctggat gacggggagg atgaggaaga tgaagacgag 1140
gcctggaagg ccttcaacgg tggctggacg gagatgcctg ggatcctgtg gatggagcct 1200
acgcagccgc ctgactttgc cctggcctat agaccgagct tcccagagga cagagagcca 1260
cagataccct acccggagcc cacctggcca cccccgctca gtgcccccag ggtcccctac 1320
cactcctcag tgctctccgt cacccggcct gtggtggtct ctgccacgca tcccacactg 1380
ccttctgccc accagcctcc tgtgatccct gccacacacc cagctttgtc ccgtgaccac 1440
cagatccccg tgatcgcagc caactatcca gatctgcctt ctgcctacca acccggtatt 1500
ctctctgtct ctcattcagc acagcctect gcccaccagc cccctatgat ctcaaccaaa 1560
tatccggagc tcttccctgc ccaccagtcc cccatgtttc cagacacccg ggtcgctggc 1620
acccagacca ccactcattt gcctggaatc ccacctaacc atgcccctct ggtcaccacc 1680
ctcggtgccc agctaccccc tcaagcccca gatgcccttg tcctcagaac ccaggccacc 1740
cagcttccca ttatcccaac tgcccagccc tctctgacca ccacctccag gtcccctgtg 1800
tctcctgccc atcaaatctc tgtgcctgct gccacccagc ccgcagccct ccccaccctc 1860
ctgccctctc agagccccac taaccagacc tcacccatca gccctacaca tccccattcc 1920
aaagcccccc aaatcccaag ggaagatggc cccagtccca agttggccct gtggctgccc 1980
tcaccagctc ccacagcagc cccaacagcc ctgggggagg ctggtcttgc cgagcacagc 2040
cagagggatg accggtggct gctggtggca ctcctggtgc caacgtgtgt ctttttggtg 2100
gtcctgcttg cactgggcat cgtgtactgc acccgctgtg gcccccatgc acccaacaag 2160
cgcatcactg actgctatcg ctgggtcatc catgctggga gcaagagccc aacagaaccc 2220
atgcccccca ggggcagcct cacaggggtg cagacctgca gaaccagcgt gggcggcagc 2280
ggcggcggcg gcagcggcgg caaaaatctg gattgttggg ttgataatga agaagatata 2340
gatgttatat taaaaaagag tacaatttta aatttagata ttaataatga tattatatca 2400
gatatatctg ggtttaattc atctgtaata acatatccag atgctcaatt ggtgcccgga 2460
ataaatggca aagcaataca tttagtaaac aatgaatctt ctgaagttat agtgcataaa 2520
gctatggata ttgaatataa tgatatgttt aataatttta ccgttagctt ttggttgagg 2580
gttcctaaag tatctgctag tcatttagaa caatatggca caaatgagta ttcaataatt 2640
agctctatga aaaaacatag tctatcaata ggatctggtt ggagtgtatc acttaaaggt 2700
aataacttaa tatggacttt aaaagattcc gcgggagaag ttagacaaat aacttttagg 2760
gatttacctg ataaatttaa tgcttattta gcaaataaat gggtttttat aactattact 2820
aatgatagat tatcttctgc taatttgtat ataaatggag tacttatggg aagtgcagaa 2880
attactggtt taggagctat tagagaggat aataatataa cattaaaact agatagatgt 2940
aataataata atcaatacgt ttctattgat aaatttagga tattttgcaa agcattaaat 3000
ccaaaagaga ttgaaaaatt atacacaagt tatttatcta taaccttttt aagagacttc 3060
tggggaaact ga 3072
Sequence Number (ID): 5
Length: 3072
Molecule Type: RNA
Features Location/Qualifiers:
- source, 1 . . . 3072
> mol type, other RNA
> organism, synthetic construct
- mRNA, 1 . . . 2274
> standard name, Human TEM1 mRNA
- misc structure, 2275 . . . 2304
> note, Linker
- mRNA, 2305 . . . 3072
> note, Tetanus Toxoid mRNA
Residues:
atgctgctgc gcctgttgct ggcctgggcg gccgcagggc ccacactggg ccaggacccc 60
tgggctgctg agccccgtgc cgcctgcggc cccagcagct gctacgctct cttcccacgg 120
cgccgcacct tcctggaggc ctggcgggcc tgccgcgagc tggggggcga cctggccact 180
cctcggaccc ccgaggaggc ccagcgtgtg gacagcctgg tgggtgcggg cccagccagc 240
cggctgctgt ggatcgggct gcagcggcag gcccggcaat gccagctgca gcgcccactg 300
cgcggcttca cgtggaccac aggggaccag gacacggctt tcaccaactg ggcccagcca 360
gcctctggag gcccctgccc ggcccagcgc tgtgtggccc tggaggcaag tggcgagcac 420
cgctggctgg agggctcgtg cacgctggct gtcgacggct acctgtgcca gtttggcttc 480
gagggcgcct gcccggcgct gcaagatgag gcgggccagg ccggcccagc cgtgtatacc 540
acgcccttcc acctggtctc cacagagttt gagtggctgc ccttcggctc tgtggccgct 600
gtgcagtgcc aggctggcag gggagcctct ctgctctgcg tgaagcagcc tgagggaggt 660
gtgggctggt cacgggctgg gcccctgtgc ctggggactg gctgcagccc tgacaacggg 720
ggctgcgaac acgaatgtgt ggaggaggtg gatggtcacg tgtcctgccg ctgcactgag 780
ggcttccggc tggcagcaga cgggcgcagt tgcgaggacc cctgtgccca ggctccgtgc 840
gagcagcagt gtgagcccgg tgggccacaa ggctacagct gccactgtcg cctgggtttc 900
cggccagcgg aggatgatcc gcaccgctgt gtggacacag atgagtgcca gattgccggt 960
gtgtgccagc agatgtgtgt caactacgtt ggtggcttcg agtgttattg tagcgaggga 1020
catgagctgg aggctgatgg catcagctgc agccctgcag gggccatggg tgcccaggct 1080
tcccaggacc tcggagatga gttgctggat gacggggagg atgaggaaga tgaagacgag 1140
gcctggaagg ccttcaacgg tggctggacg gagatgcctg ggatcctgtg gatggagcct 1200
acgcagccgc ctgactttgc cctggcctat agaccgagct tcccagagga cagagagcca 1260
cagataccct acccggagcc cacctggcca cccccgctca gtgcccccag ggtcccctac 1320
cactcctcag tgctctccgt cacccggcct gtggtggtct ctgccacgca tcccacactg 1380
ccttctgccc accagcctcc tgtgatccct gccacacacc cagctttgtc ccgtgaccac 1440
cagatccccg tgatcgcagc caactatcca gatctgcctt ctgcctacca acccggtatt 1500
ctctctgtct ctcattcagc acagcctcct gcccaccagc cccctatgat ctcaaccaaa 1560
tatccggagc tcttccctgc ccaccagtcc cccatgtttc cagacacccg ggtcgctggc 1620
acccagacca ccactcattt gcctggaatc ccacctaacc atgcccctct ggtcaccacc 1680
ctcggtgccc agctaccccc tcaagcccca gatgcccttg tcctcagaac ccaggccacc 1740
cagcttccca ttatcccaac tgcccagccc tctctgacca ccacctccag gtcccctgtg 1800
tctcctgccc atcaaatctc tgtgcctgct gccacccagc ccgcagccct ccccaccctc 1860
ctgccctctc agagccccac taaccagacc tcacccatca gccctacaca tccccattcc 1920
aaagcccccc aaatcccaag ggaagatggc cccagtccca agttggccct gtggctgccc 1980
tcaccagctc ccacagcagc cccaacagcc ctgggggagg ctggtcttgc cgagcacagc 2040
cagagggatg accggtggct gctggtggca ctcctggtgc caacgtgtgt ctttttggtg 2100
gtcctgcttg cactgggcat cgtgtactgc acccgctgtg gcccccatgc acccaacaag 2160
cgcatcactg actgctatcg ctgggtcatc catgctggga gcaagagccc aacagaaccc 2220
atgcccccca ggggcagcct cacaggggtg cagacctgca gaaccagcgt gggcggcagc 2280
ggcggcggcg gcagcggcgg caaaaatctg gattgttggg ttgataatga agaagatata 2340
gatgttatat taaaaaagag tacaatttta aatttagata ttaataatga tattatatca 2400
gatatatctg ggtttaattc atctgtaata acatatccag atgctcaatt ggtgcccgga 2460
ataaatggca aagcaataca tttagtaaac aatgaatctt ctgaagttat agtgcataaa 2520
gctatggata ttgaatataa tgatatgttt aataatttta ccgttagctt ttggttgagg 2580
gttcctaaag tatctgctag tcatttagaa caatatggca caaatgagta ttcaataatt 2640
agctctatga aaaaacatag tctatcaata ggatctggtt ggagtgtatc acttaaaggt 2700
aataacttaa tatggacttt aaaagattcc gcgggagaag ttagacaaat aacttttagg 2760
gatttacctg ataaatttaa tgcttattta gcaaataaat gggtttttat aactattact 2820
aatgatagat tatcttctgc taatttgtat ataaatggag tacttatggg aagtgcagaa 2880
attactggtt taggagctat tagagaggat aataatataa cattaaaact agatagatgt 2940
aataataata atcaatacgt ttctattgat aaatttagga tattttgcaa agcattaaat 3000
ccaaaagaga ttgaaaaatt atacacaagt tatttatcta taaccttttt aagagacttc 3060
tggggaaact ga 3072
Sequence Number (ID): 6
Length: 1023
Molecule Type: AA
Features Location/Qualifiers:
- source, 1 . . . 1023
> mol type, protein
> organism, synthetic construct
- PEPTIDE, 1 . . . 757
> note, Human TEM1 Protein
- CHAIN, 758 . . . 767
> note, Linker
- PEPTIDE, 768 . . . 1023
> note, Tetanus Toxoid
MLLRLLLAWA AAGPTLGQDP WAAEPRAACG PSSCYALFPR RRTFLEAWRA CRELGGDLAT 60
PRTPEEAQRV DSLVGAGPAS RLLWIGLORQ ARQCQLQRPL RGFTWTTGDQ DTAFTNWAQP 120
ASGGPCPAQR CVALEASGEH RWLEGSCTLA VDGYLCQFGF EGACPALQDE AGQAGPAVYT 180
TPFHLVSTEF EWLPEGSVAA VQCQAGRGAS LLCVKQPEGG VGWSRAGPLC LGTGCSPDNG 240
GCEHECVEEV DGHVSCRCTE GFRLAADGRS CEDPCAQAPC EQQCEPGGPQ GYSCHCRLGF 300
RPAEDDPHRC VDTDECQIAG VCQQMCVNYV GGFECYCSEG HELEADGISC SPAGAMGAQA 360
SQDLGDELLD DGEDEEDEDE AWKAENGGWT EMPGILWMEP TOPPDFALAY RPSFPEDREP 420
QIPYPEPTWP PPLSAPRVPY HSSVLSVTRP VVVSATHPTL PSAHOPPVIP ATHPALSRDH 480
QIPVIAANYP DLPSAYQPGI LSVSHSAQPP AHQPPMISTK YPELFPAHQS PMFPDTRVAG 540
TQTTTHLPGI PPNHAPLVTT LGAQLPPQAP DALVLRTQAT QLPIIPTAQP SLTTTSRSPV 600
SPAHQISVPA ATQPAALPTL LPSQSPTNQT SPISPTHPHS KAPQIPREDG PSPKLALWLP 660
SPAPTAAPTA LGEAGLAEHS QRDDRWLLVA LLVPTCVELV VLLALGIVYC TRCGPHAPNK 720
RITDCYRWVI HAGSKSPTEP MPPRGSLTGV QTCRTSVGGS GGGGSGGKNL DCWVDNEEDI 780
DVILKKSTIL NLDINNDIIS DISGENSSVI TYPDAQLVPG INGKAIHLVN NESSEVIVHK 840
AMDIEYNDMF NNFTVSFWLR VPKVSASHLE QYGTNEYSII SSMKKHSLSI GSGWSVSLKG 900
NNLIWTLKDS AGEVRQITER DLPDKENAYL ANKWVFITIT NDRLSSANLY INGVLMGSAE 960
ITGLGAIRED NNITLKLDRC NNNNQYVSID KFRIFCKALN PKEIEKLYTS YLSITELRDF 1020
WGN 1023
Sequence Number (ID): 7
Length: 2298
Molecule Type: DNA
Features Location/Qualifiers:
- source, 1 . . . 2298
> mol type, genomic DNA
> organism, Mus musculus
Residues:
atgctgctgc gcctgctgct ggcctgggtg gccgcggtgc ccgcactggg ccaggtcccc 60
tggacgccgg agcctcgagc cgcgtgcggc cccagcagct gctacgcgct ctttccccgg 120
cgccgcacat tcctggaagc ttggcgggcg tgccgcgaat tggggggcaa cctggccaca 180
ccgcggaccc cagaggaggc ccagcgtgtg gacagcctgg tgggggtcgg gccggccaac 240
gggctgctat ggattgggtt gcagcggcag gctaggcaat gccagccgca gcgcccactg 300
cggggcttca tatggaccac gggagaccag gacaccgcct tcaccaactg ggcccagccg 360
gctacggaag gaccctgccc agcccagcgc tgtgcagccc ttgaggccag cggagagcat 420
cgctggctcg aaggctcgtg cacactggct gtcgatggct acctctgcca gtttggtttt 480
gagggtgcct gccctgcctt gccgcttgag gtgggtcagg ccggtcccgc tgtctacacc 540
acacccttca acctggtttc cagcgagttc gaatggctgc cctttggctc cgtggcagct 600
gtgcagtgcc aagctggcag gggagcttct ctgctgtgcg tgaaacagcc ttcaggtggc 660
gtgggctggt cccagactgg cccgctgtgc ccagggactg gctgtggtcc tgacaatggg 720
ggttgcgaac atgagtgtgt ggaagaggtg gacggtgctg tgtcctgccg ctgcagtgaa 780
ggcttccgtc tagcagcaga tgggcacagt tgtgaagacc cctgtgccca ggccccctgt 840
gagcagcagt gtgaacctgg agggccacaa ggctatagct gccactgtcg ccttggcttc 900
cggccagctg aggatgatcc acaccgctgc gtggacacgg atgagtgcca gattgctggt 960
gtgtgccagc agatgtgtgt caactatgtt ggtggctttg agtgttactg cagcgagggt 1020
cacgagcttg aggcagatgg tatcagctgt agccctgcag gagccatggg tgcccaggct 1080
tcccaggatc tcagagatga gttgctggat gatggagaag aaggggagga tgaagaggag 1140
ccctgggagg actttgatgg cacctggaca gaggaacagg ggatcctatg gctggcacct 1200
acacatccac ctgactttgg cctgccctat aggcccaact tcccacagga tggagagcct 1260
cagagattgc acctggagcc tacctggcca cccccactta gtgcccccag gggcccctac 1320
cactcctcag tggtgtctgc cacacggccc atggtgatct ctgccactcg acccacacta 1380
ccttctgccc acaagacctc tgttatttca gctacacgcc cacccctgag ccctgtccac 1440
ccacctgcca tggcccctgc cacacctcca gctgtgttct ctgagcacca gatccccaaa 1500
atcaaggcca attatccaga cctgcctttt ggccacaagc ctgggataac ctcggccact 1560
cacccagcac ggtctcctcc gtaccagccc cccattatct caaccaacta tccccaagtc 1620
ttccctcccc accaggcccc tatgtctcca gatacccaca ctatcactta tttgcctcca 1680
gtcccccctc accttgatcc tggggatacc acttctaaag cccatcaaca ccctttgctc 1740
ccagatgctc caggtatcag aacccaggcc ccccagcttt ctgtctcagc tctccagccc 1800
cctcttccta ccaactccag gtcttctgtc catgaaactc ctgtgcctgc tgccaaccag 1860
cccccagcct tcccttcttc tcccctcccc cctcagaggc ccactaacca gacctcatct 1920
atcagcccta cacattccta ttccagagcc cctctagtcc caagggaagg agttcccagt 1980
cccaaatcag tgccacagct gccctcggtg ccctccacag cagctccaac agccctggca 2040
gagtcaggtc ttgcaggcca aagccaaagg gatgaccgct ggctgctggt ggcactcctg 2100
gtgccaacat gtgtcttctt ggtggtgctg cttgccctgg gcattgtgta ctgcactcgc 2160
tgtggctccc acgcacccaa caagcggatc acggactgct atcgctgggt cacacatgct 2220
gggaacaaga gctcaacaga acccatgccc cccagaggca gccttacagg ggtacagacc 2280
tgtagaacca gtgtgtga 2298
Sequence Number (ID): 8
Length: 2298
Molecule Type: RNA
Features Location/Qualifiers:
- source, 1 . . . 2298
> mol type, mRNA
> organism, Mus musculus
Residues:
atgctgctgc gcctgctgct ggcctgggtg gccgcggtgc ccgcactggg ccaggtcccc 60
tggacgccgg agcctcgagc cgcgtgcggc cccagcagct gctacgcgct ctttccccgg 120
cgccgcacat tcctggaagc ttggcgggcg tgccgcgaat tggggggcaa cctggccaca 180
ccgcggaccc cagaggaggc ccagcgtgtg gacagcctgg tgggggtcgg gccggccaac 240
gggctgctat ggattgggtt gcagcggcag gctaggcaat gccagccgca gcgcccactg 300
cggggcttca tatggaccac gggagaccag gacaccgcct tcaccaactg ggcccagccg 360
gctacggaag gaccctgccc agcccagcgc tgtgcagccc ttgaggccag cggagagcat 420
gtgcagtgcc aagctggcag gggagcttct ctgctgtgcg tgaaacagcc ttcaggtggc 660
gtgggctggt cccagactgg cccgctgtgc ccagggactg gctgtggtcc tgacaatggg 720
ggttgcgaac atgagtgtgt ggaagaggtg gacggtgctg tgtcctgccg ctgcagtgaa 780
ggcttccgtc tagcagcaga tgggcacagt tgtgaagacc cctgtgccca ggccccctgt 840
gagcagcagt gtgaacctgg agggccacaa ggctatagct gccactgtcg ccttggcttc 900
cggccagctg aggatgatcc acaccgctgc gtggacacgg atgagtgcca gattgctggt 960
gtgtgccagc agatgtgtgt caactatgtt ggtggctttg agtgttactg cagcgagggt 1020
cacgagcttg aggcagatgg tatcagctgt agccctgcag gagccatggg tgcccaggct 1080
tcccaggatc tcagagatga gttgctggat gatggagaag aaggggagga tgaagaggag 1140
ccctgggagg actttgatgg cacctggaca gaggaacagg ggatcctatg gctggcacct 1200
acacatccac ctgactttgg cctgccctat aggcccaact tcccacagga tggagagcct 1260
cagagattgc acctggagcc tacctggcca cccccactta gtgcccccag gggcccctac 1320
cactcctcag tggtgtctgc cacacggccc atggtgatct ctgccactcg acccacacta 1380
ccttctgccc acaagacctc tgttatttca gctacacgcc cacccctgag ccctgtccac 1440
ccacctgcca tggcccctgc cacacctcca gctgtgttct ctgagcacca gatccccaaa 1500
atcaaggcca attatccaga cctgcctttt ggccacaagc ctgggataac ctcggccact 1560
cacccagcac ggtctcctcc gtaccagccc cccattatct caaccaacta tccccaagtc 1620
ttccctcccc accaggcccc tatgtctcca gatacccaca ctatcactta tttgcctcca 1680
gtcccccctc accttgatcc tggggatacc acttctaaag cccatcaaca ccctttgctc 1740
ccagatgctc caggtatcag aacccaggcc ccccagcttt ctgtctcagc tctccagccc 1800
cctcttccta ccaactccag gtcttctgtc catgaaacte ctgtgcctgc tcccaaccag 1860
cccccagcct tcccttcttc tcccctcccc cctcagaggc ccactaacca gacctcatct 1920
atcagcccta cacattccta ttccagagcc cctctagtcc caagggaagg agttcccagt 1980
cccaaatcag tgccacagct gccctcggtg ccctccacag cagctccaac agccctggca 2040
gagtcaggtc ttgcaggcca aagccaaagg gatgaccgct ggctgctggt ggcactcctg 2100
gtgccaacat gtgtcttctt ggtggtgctg cttgccctgg gcattgtgta ctgcactcgc 2160
tgtggctccc acgcacccaa caagcggatc acggactgct atcgctgggt cacacatgct 2220
gggaacaaga gctcaacaga acccatgccc cccagaggca gccttacagg ggtacagacc 2280
tgtagaacca gtgtgtga 2298
Sequence Number (ID): 9
Length: 765
Molecule Type: AA
Features Location/Qualifiers:
- source, 1 . . . 765
> mol type, protein
> organism, Mus musculus
Residues:
MLLRLLLAWV AAVPALGQVP WTPEPRAACG PSSCYALFPR RRTFLEAWRA CRELGGNLAT 60
PRTPEEAQRV DSLVGVGPAN GLLWIGLORO ARQCQPORPL RGFIWTTGDQ DTAFTNWAQP 120
ATEGPCPAQR CAALEASGEH RWLEGSCTLA VDGYLCQFGF EGACPALPLE VGQAGPAVYT 180
TPFNLVSSEF EWLPEGSVAA VQCQAGRGAS LLCVKQPSGG VGWSQTGPLC PGTGCGPDNG 240
GCEHECVEEV DGAVSCRCSE GFRLAADGHS CEDPCAQAPC EQQCEPGGPQ GYSCHCRLGF 300
RPAEDDPHRC VDTDECQIAG VCQQMCVNYV GGFECYCSEG HELEADGISC SPAGAMGAQA 360
SQDLRDELLD DGEEGEDEEE PWEDEDGTWT EEQGILWLAP THPPDFGLPY RPNFPQDGEP 420
QRLHLEPTWP PPLSAPRGPY HSSVVSATRP MVISATRPTL PSAHKTSVIS ATRPPLSPVH 480
PPAMAPATPP AVESEHQIPK IKANYPDLPF GHKPGITSAT HPARSPPYQP PIISTNYPQV 540
FPPHQAPMSP DTHTITYLPP VPPHLDPGDT TSKAHQHPLL PDAPGIRTQA PQLSVSALQP 600
PLPTNSRSSV HETPVPAANQ PPAFPSSPLP PQRPTNQTSS ISPTHSYSRA PLVPREGVPS 660
PKSVPQLPSV PSTAAPTALA ESGLAGQSQR DDRWLLVALL VPTCVELVVL LALGIVYCTR 720
CGSHAPNKRI TDCYRWVTHA GNKSSTEPMP PRGSLTGVQT CRTSV 765
Sequence Number (ID): 10
Length: 3096
Molecule Type: DNA
Features Location/Qualifiers:
- source, 1 . . . 3096
> mol type, other DNA
> organism, synthetic construct
- gene, 1 . . . 2298
> gene, Mouse TEM1
- misc feature, 2299 . . . 2328
> note, Linker
- gene, 2329 . . . 3096
> gene, Tetanus Toxoid
Residues:
atgctgctgc gcctgctgct ggcctgggtg gccgcggtgc ccgcactggg ccaggtcccc 60
tggacgccgg agcctcgagc cgcgtgcggc cccagcagct gctacgcgct ctttccccgg 120
cgccgcacat tcctggaagc ttggcgggcg tgccgcgaat tggggggcaa cctggccaca 180
ccgcggaccc cagaggaggc ccagcgtgtg gacagcctgg tgggggtcgg gccggccaac 240
gggctgctat ggattgggtt gcagcggcag gctaggcaat gccagccgca gcgcccactg 300
cggggcttca tatggaccac gggagaccag gacaccgcct tcaccaactg ggcccagccg 360
gctacggaag gaccctgccc agcccagcgc tgtgcagccc ttgaggccag cggagagcat 420
cgctggctcg aaggctcgtg cacactggct gtcgatggct acctctgcca gtttggtttt 480
gagggtgcct gccctgcctt gccgcttgag gtgggtcagg ccggtcccgc tgtctacacc 540
acacccttca acctggtttc cagcgagttc gaatggctgc cctttggctc cgtggcagct 600
gtgcagtgcc aagctggcag gggagcttct ctgctgtgcg tgaaacagcc ttcaggtggc 660
gtgggctggt cccagactgg cccgctgtgc ccagggactg gctgtggtcc tgacaatggg 720
ggttgcgaac atgagtgtgt ggaagaggtg gacggtgctg tgtcctgccg ctgcagtgaa 780
ggcttccgtc tagcagcaga tgggcacagt tgtgaagacc cctgtgccca ggccccctgt 840
gagcagcagt gtgaacctgg agggccacaa ggctatagct gccactgtcg ccttggcttc 900
cggccagctg aggatgatcc acaccgctgc gtggacacgg atgagtgcca gattgctggt 960
gtgtgccagc agatgtgtgt caactatgtt ggtggctttg agtgttactg cagcgagggt 1020
cacgagcttg aggcagatgg tatcagctgt agccctgcag gagccatggg tgcccaggct 1080
tcccaggatc tcagagatga gttgctggat gatggagaag aaggggagga tgaagaggag 1140
ccctgggagg actttgatgg cacctggaca gaggaacagg ggatcctatg gctggcacct 1200
acacatccac ctgactttgg cctgccctat aggcccaact tcccacagga tggagagcct 1260
cagagattgc acctggagcc tacctggcca cccccactta gtgcccccag gggcccctac 1320
cactcctcag tggtgtctgc cacacggccc atggtgatct ctgccactcg acccacacta 1380
ccttctgccc acaagacctc tgttatttca gctacacgcc cacccctgag ccctgtccac 1440
ccacctgcca tggcccctgc cacacctcca gctgtgttct ctgagcacca gatccccaaa 1500
atcaaggcca attatccaga cctgcctttt ggccacaagc ctgggataac ctcggccact 1560
cacccagcac ggtctcctcc gtaccagccc cccattatct caaccaacta tccccaagtc 1620
ttccctcccc accaggcccc tatgtctcca gatacccaca ctatcactta tttgcctcca 1680
gtcccccctc accttgatcc tggggatacc acttctaaag cccatcaaca ccctttgctc 1740
ccagatgctc caggtatcag aacccaggcc ccccagcttt ctgtctcagc tctccagccc 1800
cctcttccta ccaactccag gtcttctgtc catgaaactc ctgtgcctgc tgccaaccag 1860
cccccagcct tcccttcttc tcccctcccc cctcagaggc ccactaacca gacctcatct 1920
atcagcccta cacattccta ttccagagcc cctctagtcc caagggaagg agttcccagt 1980
cccaaatcag tgccacagct gccctcggtg ccctccacag cagctccaac agccctggca 2040
gagtcaggtc ttgcaggcca aagccaaagg gatgaccgct ggctgctggt ggcactcctg 2100
gtgccaacat gtgtcttctt ggtggtgctg cttgccctgg gcattgtgta ctgcactcgc 2160
tgtggctccc acgcacccaa caagcggatc acggactgct atcgctgggt cacacatgct 2220
gggaacaaga gctcaacaga acccatgccc cccagaggca gccttacagg ggtacagacc 2280
tgtagaacca gtgtgggcgg cagcggcggc ggcggcagcg gcggcaaaaa tctggattgt 2340
tgggttgata atgaagaaga tatagatgtt atattaaaaa agagtacaat tttaaattta 2400
gatattaata atgatattat atcagatata tctgggttta attcatctgt aataacatat 2460
ccagatgctc aattggtgcc cggaataaat ggcaaagcaa tacatttagt aaacaatgaa 2520
tcttctgaag ttatagtgca taaagctatg gatattgaat ataatgatat gtttaataat 2580
tttaccgtta gcttttggtt gagggttcct aaagtatctg ctagtcattt agaacaatat 2640
ggcacaaatg agtattcaat aattagctct atgaaaaaac atagtctatc aataggatct 2700
ggttggagtg tatcacttaa aggtaataac ttaatatgga ctttaaaaga ttccgcggga 2760
gaagttagac aaataacttt tagggattta cctgataaat ttaatgctta tttagcaaat 2820
aaatgggttt ttataactat tactaatgat agattatctt ctgctaattt gtatataaat 2880
ggagtactta tgggaagtgc agaaattact ggtttaggag ctattagaga ggataataat 2940
ataacattaa aactagatag atgtaataat aataatcaat acgtttctat tgataaattt 3000
aggatatttt gcaaagcatt aaatccaaaa gagattgaaa aattatacac aagttattta 3060
tctataacct ttttaagaga cttctgggga aactga 3096
Sequence Number (ID): 11
Length: 3096
Molecule Type: RNA
Features Location/Qualifiers:
- source, 1 . . . 3096
> mol type, other RNA
> organism, Synthetic construct
- mRNA, 1 . . . 2298
> gene, Mouse TEM1 mRNA
- misc feature, 2299 . . . 2328
> note, Linker
- gene, 2329 . . . 3096
> gene, Tetanus Toxoid mRNA
atgctgctgc gcctgctgct ggcctgggtg gccgcggtgc ccgcactggg ccaggtcccc 60
tggacgccgg agcctcgagc cgcgtgcggc cccagcagct gctacgcgct ctttccccgg 120
cgccgcacat tcctggaagc ttggcgggcg tgccgcgaat tggggggcaa cctggccaca 180
ccgcggaccc cagaggaggc ccagcgtgtg gacagcctgg tgggggtcgg gccggccaac 240
gggctgctat ggattgggtt gcagcggcag gctaggcaat gccagccgca gcgcccactg 300
cggggcttca tatggaccac gggagaccag gacaccgcct tcaccaactg ggcccagccg 360
gctacggaag gaccctgccc agcccagcgc tgtgcagccc ttgaggccag cggagagcat 420
cgctggctcg aaggctcgtg cacactggct gtcgatggct acctctgcca gtttggtttt 480
gagggtgcct gccctgcctt gccgcttgag gtgggtcagg ccggtcccgc tgtctacacc 540
acacccttca acctggtttc cagcgagttc gaatggctgc cctttggctc cgtggcagct 600
gtgcagtgcc aagctggcag gggagcttct ctgctgtgcg tgaaacagcc ttcaggtggc 660
gtgggctggt cccagactgg cccgctgtgc ccagggactg gctgtggtcc tgacaatggg 720
ggttgcgaac atgagtgtgt ggaagaggtg gacggtgctg tgtcctgccg ctgcagtgaa 780
ggcttccgtc tagcagcaga tgggcacagt tgtgaagacc cctgtgccca ggccccctgt 840
gagcagcagt gtgaacctgg agggccacaa ggctatagct gccactgtcg ccttggcttc 900
cggccagctg aggatgatcc acaccgctgc gtggacacgg atgagtgcca gattgctggt 960
gtgtgccagc agatgtgtgt caactatgtt ggtggctttg agtgttactg cagcgagggt 1020
cacgagcttg aggcagatgg tatcagctgt agccctgcag gagccatggg tgcccaggct 1080
tcccaggatc tcagagatga gttgctggat gatggagaag aaggggagga tgaagaggag 1140
ccctgggagg actttgatgg cacctggaca gaggaacagg ggatcctatg gctggcacct 1200
acacatccac ctgactttgg cctgccctat aggcccaact tcccacagga tggagagcct 1260
cagagattgc acctggagcc tacctggcca cccccactta gtgcccccag gggcccctac 1320
cactcctcag tggtgtctgc cacacggccc atggtgatct ctgccactcg acccacacta 1380
ccttctgccc acaagacctc tgttatttca gctacacgcc cacccctgag ccctgtccac 1440
ccacctgcca tggcccctgc cacacctcca gctgtgttct ctgagcacca gatccccaaa 1500
atcaaggcca attatccaga cctgcctttt ggccacaagc ctgggataac ctcggccact 1560
cacccagcac ggtctcctcc gtaccagccc cccattatct caaccaacta tccccaagtc 1620
ttccctcccc accaggcccc tatgtctcca gatacccaca ctatcactta tttgcctcca 1680
gtcccccctc accttgatcc tggggatacc acttctaaag cccatcaaca ccctttgctc 1740
ccagatgctc caggtatcag aacccaggcc ccccagcttt ctgtctcagc tctccagccc 1800
cctcttccta ccaactccag gtcttctgtc catgaaactc ctgtgcctgc tgccaaccag 1860
cccccagcct tcccttcttc tcccctcccc cctcagaggc ccactaacca gacctcatct 1920
atcagcccta cacattccta ttccagagcc cctctagtcc caagggaagg agttcccagt 1980
cccaaatcag tgccacagct gccctcggtg ccctccacag cagctccaac agccctggca 2040
gagtcaggtc ttgcaggcca aagccaaagg gatgaccgct ggctgctggt ggcactcctg 2100
gtgccaacat gtgtcttctt ggtggtgctg cttgccctgg gcattgtgta ctgcactcgc 2160
tgtggctccc acgcacccaa caagcggatc acggactgct atcgctgggt cacacatgct 2220
gggaacaaga gctcaacaga acccatgccc cccagaggca gccttacagg ggtacagacc 2280
tgtagaacca gtgtgggcgg cagcggcggc ggcggcagcg gcggcaaaaa tctggattgt 2340
tgggttgata atgaagaaga tatagatgtt atattaaaaa agagtacaat tttaaattta 2400
gatattaata atgatattat atcagatata tctgggttta attcatctgt aataacatat 2460
ccagatgctc aattggtgcc cggaataaat ggcaaagcaa tacatttagt aaacaatgaa 2520
tcttctgaag ttatagtgca taaagctatg gatattgaat ataatgatat gtttaataat 2580
tttaccgtta gcttttggtt gagggttcct aaagtatctg ctagtcattt agaacaatat 2640
ggcacaaatg agtattcaat aattagctct atgaaaaaac atagtctatc aataggatct 2700
ggttggagtg tatcacttaa aggtaataac ttaatatgga ctttaaaaga ttccgcggga 2760
gaagttagac aaataacttt tagggattta cctgataaat ttaatgctta tttagcaaat 2820
aaatgggttt ttataactat tactaatgat agattatctt ctgctaattt gtatataaat 2880
ggagtactta tgggaagtgc agaaattact ggtttaggag ctattagaga ggataataat 2940
ataacattaa aactagatag atgtaataat aataatcaat acgtttctat tgataaattt 3000
aggatatttt gcaaagcatt aaatccaaaa gagattgaaa aattatacac aagttattta 3060
tctataacct ttttaagaga cttctgggga aactga 3096
Sequence Number (ID): 12
Length: 1031
Molecule Type: AA
Features Location/Qualifiers:
- source, 1 . . . 1031
> mol type, protein
> organism, Synthetic construct
- PEPTIDE, 1 . . . 765
> note, Mouse TEM1
- CHAIN, 766 . . . 775
> note, Linker
- PEPTIDE, 776 . . . 1031
> note, Tetanus Toxoid
Residues:
MLLRLLLAWV AAVPALGQVP WTPEPRAACG PSSCYALFPR RRTFLEAWRA CRELGGNLAT 60
PRTPEEAQRV DSLVGVGPAN GLLWIGLORQ ARQCQPORPL RGFIWTTGDQ DTAFTNWAQP 120
ATEGPCPAQR CAALEASGEH RWLEGSCTLA VDGYLCQFGF EGACPALPLE VGQAGPAVYT 180
TPENLVSSEF EWLPEGSVAA VQCQAGRGAS LLCVKQPSGG VGWSQTGPLC PGTGCGPDNG 240
GCEHECVEEV DGAVSCRCSE GFRLAADGHS CEDPCAQAPC EQQCEPGGPQ GYSCHCRLGF 300
RPAEDDPHRC VDTDECQIAG VCQQMCVNYV GGFECYCSEG HELEADGISC SPAGAMGAQA 360
SQDLRDELLD DGEEGEDEEE PWEDEDGTWT EEQGILWLAP THPPDFGLPY RPNFPQDGEP 420
QRLHLEPTWP PPLSAPRGPY HSSVVSATRP MVISATRPTL PSAHKTSVIS ATRPPLSPVH 480
PPAMAPATPP AVESEHQIPK IKANYPDLPF GHKPGITSAT HPARSPPYQP PIISTNYPQV 540
FPPHQAPMSP DTHTITYLPP VPPHLDPGDT TSKAHQHPLL PDAPGIRTQA PQLSVSALQP 600
PLPTNSRSSV HETPVPAANQ PPAFPSSPLP PQRPTNQTSS ISPTHSYSRA PLVPREGVPS 660
PKSVPQLPSV PSTAAPTALA ESGLAGQSQR DDRWLLVALL VPTCVELVVL LALGIVYCTR 720
CGSHAPNKRI TDCYRWVTHA GNKSSTEPMP PRGSLTGVQT CRTSVGGSGG GGSGGKNLDC 780
WVDNEEDIDV ILKKSTILNL DINNDIISDI SGENSSVITY PDAQLVPGIN GKAIHLVNNE 840
SSEVIVHKAM DIEYNDMENN FTVSEWLRVP KVSASHLEQY GTNEYSIISS MKKHSLSIGS 900
GWSVSLKGNN LIWTLKDSAG EVRQITERDL PDKENAYLAN KWVFITITND RLSSANLYIN 960
GVLMGSAEIT GLGAIREDNN ITLKLDRCNN NNQYVSIDKF RIFCKALNPK EIEKLYTSYL 1020
SITFLRDEWG N 1031
Sequence Number (ID): 13
Length: 56
Molecule Type: DNA
Features Location/Qualifiers:
- source, 1 . . . 56
> mol type, other DNA
> organism, Synthetic construct
Residues:
acatttgctt ctgacacaac tgtgttcact agcaacctca aacagacacc gccacc 56
Sequence Number (ID): 14
Length: 264
Molecule Type: DNA
Features Location/Qualifiers:
- source, 1 . . . 264
> mol_type, other DNA
> organism, Synthetic construct
Residues:
gctcgctttc ttgctgtcca atttctatta aaggttcctt tgttccctaa gtccaactac 60
taaactgggg gatattatga agggccttga gcatctggat tctgcctaat aaaaaacatt 120
tattttcatt gcgctcgctt tcttgctgtc caatttctat taaaggttcc tttgttccct 180
aagtccaact actaaactgg gggatattat gaagggcctt gagcatctgg attctgccta 240
ataaaaaaca tttattttca ttgc 264
Sequence Number (ID): 15
Length: 105
Molecule Type: DNA
Features Location/Qualifiers:
- source, 1 . . . 105
> mol type, other DNA
> organism, Synthetic construct
Residues:
aaaaaaaaaa aaaaaaaaaa aaaaaaaaaa aaaaaaaaaa aaaaaaaaaa aaaaaaaaaa 60
aaaaaaaaaa aaaaaaaaaa aaaaaaaaaa aaaaaaaaag agacg 105
Sequence Number (ID): 16
Length: 2699
Molecule Type: DNA
Features Location/Qualifiers:
- source, 1 . . . 2699
> mol type, other DNA
> organism, Synthetic construct
- 5'UTR, 1 . . . 56
> note, 5'-UTR
- gene, 57 . . . 2330
> gene, hTEM1
- 3'UTR, 2331 . . . 2594
> note, 3'-UTR
- polyA site, 2595 . . . 2699
> note, Poly-A tail
Residues:
acatttgctt ctgacacaac tgtgttcact agcaacctca aacagacacc gccaccatgc 60
tgctgcgcct gttgctggcc tgggcggccg cagggcccac actgggccag gacccctggg 120
ctgctgagcc ccgtgccgcc tgcggcccca gcagctgcta cgctctcttc ccacggcgcc 180
gcaccttcct ggaggcctgg cgggcctgcc gcgagctggg gggcgacctg gccactcctc 240
ggacccccga ggaggcccag cgtgtggaca gcctggtggg tgcgggccca gccagccggc 300
tgctgtggat cgggctgcag cggcaggccc ggcaatgcca gctgcagcgc ccactgcgcg 360
gcttcacgtg gaccacaggg gaccaggaca cggctttcac caactgggcc cagccagcct 420
ctggaggccc ctgcccggcc cagcgctgtg tggccctgga ggcaagtggc gagcaccgct 480
ggctggaggg ctcgtgcacg ctggctgtcg acggctacct gtgccagttt ggcttcgagg 540
gcgcctgccc ggcgctgcaa gatgaggcgg gccaggccgg cccagccgtg tataccacgc 600
ccttccacct ggtctccaca gagtttgagt ggctgccctt cggctctgtg gccgctgtgc 660
agtgccaggc tggcagggga gcctctctgc tctgcgtgaa gcagcctgag ggaggtgtgg 720
gctggtcacg ggctgggccc ctgtgcctgg ggactggctg cagccctgac aacgggggct 780
gcgaacacga atgtgtggag gaggtggatg gtcacgtgtc ctgccgctgc actgagggct 840
tccggctggc agcagacggg cgcagttgcg aggacccctg tgcccaggct ccgtgcgagc 900
agcagtgtga gcccggtggg ccacaaggct acagctgcca ctgtcgcctg ggtttccggc 960
cagcggagga tgatccgcac cgctgtgtgg acacagatga gtgccagatt gccggtgtgt 1020
gccagcagat gtgtgtcaac tacgttggtg gcttcgagtg ttattgtagc gagggacatg 1080
agctggaggc tgatggcatc agctgcagcc ctgcaggggc catgggtgcc caggcttccc 1140
aggacctcgg agatgagttg ctggatgacg gggaggatga ggaagatgaa gacgaggcct 1200
ggaaggcctt caacggtggc tggacggaga tgcctgggat cctgtggatg gagcctacgc 1260
agccgcctga ctttgccctg gcctatagac cgagcttccc agaggacaga gagccacaga 1320
taccctaccc ggagcccacc tggccacccc cgctcagtgc ccccagggtc ccctaccact 1380
cctcagtgct ctccgtcacc cggcctgtgg tggtctctgc cacgcatccc acactgcctt 1440
ctgcccacca gcctcctgtg atccctgcca cacacccagc tttgtcccgt gaccaccaga 1500
tccccgtgat cgcagccaac tatccagatc tgccttctgc ctaccaaccc ggtattctct 1560
ctgtctctca ttcagcacag cctcctgccc accagccccc tatgatctca accaaatatc 1620
cggagctctt ccctgcccac cagtccccca tgtttccaga cacccgggtc gctggcaccc 1680
agaccaccac tcatttgcct ggaatcccac ctaaccatgc ccctctggtc accaccctcg 1740
gtgcccagct accccctcaa gccccagatg cccttgtcct cagaacccag gccacccagc 1800
ttcccattat cccaactgcc cagccctctc tgaccaccac ctccaggtcc cctgtgtctc 1860
ctgcccatca aatctctgtg cctgctgcca cccagcccgc agccctcccc accctcctgc 1920
cctctcagag ccccactaac cagacctcac ccatcagccc tacacatccc cattccaaag 1980
ccccccaaat cccaagggaa gatggcccca gtcccaagtt ggccctgtgg ctgccctcac 2040
cagctcccac agcagcccca acagccctgg gggaggctgg tcttgccgag cacagccaga 2100
gggatgaccg gtggctgctg gtggcactcc tggtgccaac gtgtgtcttt ttggtggtcc 2160
tgcttgcact gggcatcgtg tactgcaccc gctgtggccc ccatgcaccc aacaagcgca 2220
tcactgactg ctatcgctgg gtcatccatg ctgggagcaa gagcccaaca gaacccatgc 2280
cccccagggg cagcctcaca ggggtgcaga cctgcagaac cagcgtgtga gctcgctttc 2340
ttgctgtcca atttctatta aaggttcctt tgttccctaa gtccaactac taaactgggg 2400
gatattatga agggccttga gcatctggat tctgcctaat aaaaaacatt tattttcatt 2460
gcgctcgctt tcttgctgtc caatttctat taaaggttcc tttgttccct aagtccaact 2520
actaaactgg gggatattat gaagggcctt gagcatctgg attctgccta ataaaaaaca 2580
tttattttca ttgcaaaaaa aaaaaaaaaa aaaaaaaaaa aaaaaaaaaa aaaaaaaaaa 2640
aaaaaaaaaa aaaaaaaaaa aaaaaaaaaa aaaaaaaaaa aaaaaaaaaa aaagagacg 2699
Sequence Number (ID): 17
Length: 3497
Molecule Type: DNA
Features Location/Qualifiers:
- source, 1 .. 3497
> mol type, other DNA
> organism, Synthetic construct
Residues:
acatttgctt ctgacacaac tgtgttcact agcaacctca aacagacacc gccaccatgc 60
tgctgcgcct gttgctggcc tgggcggccg cagggcccac actgggccag gacccctggg 120
ctgctgagcc ccgtgccgcc tgcggcccca gcagctgcta cgctctcttc ccacggcgcc 180
gcaccttcct ggaggcctgg cgggcctgcc gcgagctggg gggcgacctg gccactcctc 240
ggacccccga ggaggcccag cgtgtggaca gcctggtggg tgcgggccca gccagccggc 300
tgctgtggat cgggctgcag cggcaggccc ggcaatgcca gctgcagcgc ccactgcgcg 360
gcttcacgtg gaccacaggg gaccaggaca cggctttcac caactgggcc cagccagcct 420
ctggaggccc ctgcccggcc cagcgctgtg tggccctgga ggcaagtggc gagcaccgct 480
ggctggaggg ctcgtgcacg ctggctgtcg acggctacct gtgccagttt ggcttcgagg 540
gcgcctgccc ggcgctgcaa gatgaggcgg gccaggccgg cccagccgtg tataccacgc 600
ccttccacct ggtctccaca gagtttgagt ggctgccctt cggctctgtg gccgctgtgc 660
agtgccaggc tggcagggga gcctctctgc tctgcgtgaa gcagcctgag ggaggtgtgg 720
gctggtcacg ggctgggccc ctgtgcctgg ggactggctg cagccctgac aacgggggct 780
gcgaacacga atgtgtggag gaggtggatg gtcacgtgtc ctgccgctgc actgagggct 840
tccggctggc agcagacggg cgcagttgcg aggacccctg tgcccaggct ccgtgcgagc 900
agcagtgtga gcccggtggg ccacaaggct acagctgcca ctgtcgcctg ggtttccggc 960
cagcggagga tgatccgcac cgctgtgtgg acacagatga gtgccagatt gccggtgtgt 1020
gccagcagat gtgtgtcaac tacgttggtg gcttcgagtg ttattgtagc gagggacatg 1080
agctggaggc tgatggcatc agctgcagcc ctgcaggggc catgggtgcc caggcttccc 1140
aggacctcgg agatgagttg ctggatgacg gggaggatga ggaagatgaa gacgaggcct 1200
ggaaggcctt caacggtggc tggacggaga tgcctgggat cctgtggatg gagcctacgc 1260
agccgcctga ctttgccctg gcctatagac cgagcttccc agaggacaga gagccacaga 1320
taccctaccc ggagcccacc tggccacccc cgctcagtgc ccccagggtc ccctaccact 1380
cctcagtgct ctccgtcacc cggcctgtgg tggtctctgc cacgcatccc acactgcctt 1440
ctgcccacca gcctcctgtg atccctgcca cacacccagc tttgtcccgt gaccaccaga 1500
tccccgtgat cgcagccaac tatccagatc tgccttctgc ctaccaaccc ggtattctct 1560
ctgtctctca ttcagcacag cctcctgccc accagccccc tatgatctca accaaatatc 1620
cggagctctt ccctgcccac cagtccccca tgtttccaga cacccgggtc gctggcaccc 1680
agaccaccac tcatttgcct ggaatcccac ctaaccatgc ccctctggtc accaccctcg 1740
gtgcccagct accccctcaa gccccagatg cccttgtcct cagaacccag gccacccagc 1800
ttcccattat cccaactgcc cagccctctc tgaccaccac ctccaggtcc cctgtgtctc 1860
ctgcccatca aatctctgtg cctgctgcca cccagcccgc agccctcccc accctcctgc 1920
cctctcagag ccccactaac cagacctcac ccatcagccc tacacatccc cattccaaag 1980
ccccccaaat cccaagggaa gatggcccca gtcccaagtt ggccctgtgg ctgccctcac 2040
cagctcccac agcagcccca acagccctgg gggaggctgg tcttgccgag cacagccaga 2100
gggatgaccg gtggctgctg gtggcactcc tggtgccaac gtgtgtcttt ttggtggtcc 2160
tgcttgcact gggcatcgtg tactgcaccc gctgtggccc ccatgcaccc aacaagcgca 2220
tcactgactg ctatcgctgg gtcatccatg ctgggagcaa gagcccaaca gaacccatgc 2280
cccccagggg cagcctcaca ggggtgcaga cctgcagaac cagcgtgggc ggcagcggcg 2340
gcggcggcag cggcggcaaa aatctggatt gttgggttga taatgaagaa gatatagatg 2400
ttatattaaa aaagagtaca attttaaatt tagatattaa taatgatatt atatcagata 2460
tatctgggtt taattcatct gtaataacat atccagatgc tcaattggtg cccggaataa 2520
atggcaaagc aatacattta gtaaacaatg aatcttctga agttatagtg cataaagcta 2580
tggatattga atataatgat atgtttaata attttaccgt tagcttttgg ttgagggttc 2640
ctaaagtatc tgctagtcat ttagaacaat atggcacaaa tgagtattca ataattagct 2700
ctatgaaaaa acatagtcta tcaataggat ctggttggag tgtatcactt aaaggtaata 2760
acttaatatg gactttaaaa gattccgcgg gagaagttag acaaataact tttagggatt 2820
tacctgataa atttaatgct tatttagcaa ataaatgggt ttttataact attactaatg 2880
atagattatc ttctgctaat ttgtatataa atggagtact tatgggaagt gcagaaatta 2940
ctggtttagg agctattaga gaggataata atataacatt aaaactagat agatgtaata 3000
ataataatca atacgtttct attgataaat ttaggatatt ttgcaaagca ttaaatccaa 3060
aagagattga aaaattatac acaagttatt tatctataac ctttttaaga gacttctggg 3120
gaaactgagc tcgctttctt gctgtccaat ttctattaaa ggttcctttg ttccctaagt 3180
ccaactacta aactggggga tattatgaag ggccttgagc atctggattc tgcctaataa 3240
aaaacattta ttttcattgc gctcgctttc ttgctgtcca atttctatta aaggttcctt 3300
tgttccctaa gtccaactac taaactgggg gatattatga agggccttga gcatctggat 3360
tctgcctaat aaaaaacatt tattttcatt gcaaaaaaaa aaaaaaaaaa aaaaaaaaaa 3420
aaaaaaaaaa aaaaaaaaaa aaaaaaaaaa aaaaaaaaaa aaaaaaaaaa aaaaaaaaaa 3480
aaaaaaaaaa agagacg 3497
Sequence Number (ID): 18
Length: 2723
Molecule Type: DNA
Features Location/Qualifiers:
- source, 1 . . . 2723
> mol type, other DNA
> organism, Synthetic construct
Residues:
acatttgctt ctgacacaac tgtgttcact agcaacctca aacagacacc gccaccatgc 60
tgctgcgcct gctgctggcc tgggtggccg cggtgcccgc actgggccag gtcccctgga 120
cgccggagcc tcgagccgcg tgcggcccca gcagctgcta cgcgctcttt ccccggcgcc 180
gcacattcct ggaagcttgg cgggcgtgcc gcgaattggg gggcaacctg gccacaccgc 240
ggaccccaga ggaggcccag cgtgtggaca gcctggtggg ggtcgggccg gccaacgggc 300
tgctatggat tgggttgcag cggcaggcta ggcaatgcca gccgcagcgc ccactgcggg 360
gcttcatatg gaccacggga gaccaggaca ccgccttcac caactgggcc cagccggcta 420
cggaaggacc ctgcccagcc cagcgctgtg cagcccttga ggccagcgga gagcatcgct 480
ggctcgaagg ctcgtgcaca ctggctgtcg atggctacct ctgccagttt ggttttgagg 540
gtgcctgccc tgccttgccg cttgaggtgg gtcaggccgg tcccgctgtc tacaccacac 600
ccttcaacct ggtttccagc gagttcgaat ggctgccctt tggctccgtg gcagctgtgc 660
agtgccaagc tggcagggga gcttctctgc tgtgcgtgaa acagccttca ggtggcgtgg 720
gctggtccca gactggcccg ctgtgcccag ggactggctg tggtcctgac aatgggggtt 780
gcgaacatga gtgtgtggaa gaggtggacg gtgctgtgtc ctgccgctgc agtgaaggct 840
tccgtctagc agcagatggg cacagttgtg aagacccctg tgcccaggcc ccctgtgagc 900
agcagtgtga acctggaggg ccacaaggct atagctgcca ctgtcgcctt ggcttccggc 960
cagctgagga tgatccacac cgctgcgtgg acacggatga gtgccagatt gctggtgtgt 1020
gccagcagat gtgtgtcaac tatgttggtg gctttgagtg ttactgcagc gagggtcacg 1080
agcttgaggc agatggtatc agctgtagcc ctgcaggagc catgggtgcc caggcttccc 1140
aggatctcag agatgagttg ctggatgatg gagaagaagg ggaggatgaa gaggagccct 1200
gggaggactt tgatggcacc tggacagagg aacaggggat cctatggctg gcacctacac 1260
atccacctga ctttggcctg ccctataggc ccaacttccc acaggatgga gagcctcaga 1320
gattgcacct ggagcctacc tcgccacccc cacttagtgc ccccaggggc ccctaccact 1380
cctcagtggt gtctgccaca cggcccatgg tgatctctgc cactcgaccc acactacctt 1440
ctgcccacaa gacctctgtt atttcagcta cacgcccacc cctgagccct gtccacccac 1500
ctgccatggc ccctgccaca cctccagctg tgttctctga gcaccagatc cccaaaatca 1560
aggccaatta tccagacctg ccttttggcc acaagcctgg gataacctcg gccactcacc 1620
cagcacggtc tcctccgtac cagcccccca ttatctcaac caactatccc caagtcttcc 1680
ctccccacca ggcccctatg tctccagata cccacactat cacttatttg cctccagtcc 1740
cccctcacct tgatcctggg gataccactt ctaaagccca tcaacaccct ttgctcccag 1800
atgctccagg tatcagaacc caggcccccc agctttctgt ctcagctctc cagccccctc 1860
ttcctaccaa ctccaggtct tctgtccatg aaactcctgt gcctgctgcc aaccagcccc 1920
cagccttccc ttcttctccc ctcccccctc agaggcccac taaccagacc tcatctatca 1980
gccctacaca ttcctattcc agagcccctc tagtcccaag ggaaggagtt cccagtccca 2040
aatcagtgcc acagctgccc tcggtgccct ccacagcage tccaacagcc ctggcagagt 2100
caggtcttgc aggccaaagc caaagggatg accgctggct gctggtggca ctcctggtgc 2160
caacatgtgt cttcttggtg gtgctgcttg ccctgggcat tgtgtactgc actcgctgtg 2220
gctcccacgc acccaacaag cggatcacgg actgctatcg ctgggtcaca catgctggga 2280
acaagagctc aacagaaccc atgcccccca gaggcagcct tacaggggta cagacctgta 2340
gaaccagtgt gtgagctcgc tttcttgctg tccaatttct attaaaggtt cctttgttcc 2400
ctaagtccaa ctactaaact gggggatatt atgaagggcc ttgagcatct ggattctgcc 2460
taataaaaaa catttatttt cattgcgctc gctttcttgc tgtccaattt ctattaaagg 2520
ttcctttgtt ccctaagtcc aactactaaa ctgggggata ttatgaaggg ccttgagcat 2580
ctggattctg cctaataaaa aacatttatt ttcattgcaa aaaaaaaaaa aaaaaaaaaa 2640
aaaaaaaaaa aaaaaaaaaa aaaaaaaaaa aaaaaaaaaa aaaaaaaaaa aaaaaaaaaa 2700
aaaaaaaaaa aaaaaaagag acg 2723
Sequence Number (ID): 19
Length: 3521
Molecule Type: DNA
Features Location/Qualifiers:
- source, 1 . . . 3521
> mol type, other DNA
> organism, Synthetic construct
Residues:
acatttgctt ctgacacaac tgtgttcact agcaacctca aacagacacc gccaccatgc 60
tgctgcgcct gctgctggcc tgggtggccg cggtgcccgc actgggccag gtcccctgga 120
cgccggagcc tcgagccgcg tgcggcccca gcagctgcta cgcgctcttt ccccggcgcc 180
gcacattcct ggaagcttgg cgggcgtgcc gcgaattggg gggcaacctg gccacaccgc 240
ggaccccaga ggaggcccag cgtgtggaca gcctggtggg ggtcgggccg gccaacgggc 300
tgctatggat tgggttgcag cggcaggcta ggcaatgcca gccgcagcgc ccactgcggg 360
gcttcatatg gaccacggga gaccaggaca ccgccttcac caactgggcc cagccggcta 420
cggaaggacc ctgcccagcc cagcgctgtg cagcccttga ggccagcgga gagcatcgct 480
ggctcgaagg ctcgtgcaca ctggctgtcg atggctacct ctgccagttt ggttttgagg 540
gtgcctgccc tgccttgccg cttgaggtgg gtcaggccgg tcccgctgtc tacaccacac 600
ccttcaacct ggtttccagc gagttcgaat ggctgccctt tggctccgtg gcagctgtgc 660
agtgccaagc tggcagggga gcttctctgc tgtgcgtgaa acagccttca ggtggcgtgg 720
gctggtccca gactggcccg ctgtgcccag ggactggctg tggtcctgac aatgggggtt 780
gcgaacatga gtgtgtggaa gaggtggacg gtgctgtgtc ctgccgctgc agtgaaggct 840
tccgtctagc agcagatggg cacagttgtg aagacccctg tgcccaggcc ccctgtgagc 900
agcagtgtga acctggaggg ccacaaggct atagctgcca ctgtcgcctt ggcttccggc 960
cagctgagga tgatccacac cgctgcgtgg acacggatga gtgccagatt gctggtgtgt 1020
gccagcagat gtgtgtcaac tatgttggtg gctttgagtg ttactgcagc gagggtcacg 1080
agcttgaggc agatggtatc agctgtagcc ctgcaggagc catgggtgcc caggcttccc 1140
aggatctcag agatgagttg ctggatgatg gagaagaagg ggaggatgaa gaggagccct 1200
gggaggactt tgatggcacc tggacagagg aacaggggat cctatggctg gcacctacac 1260
atccacctga ctttggcctg ccctataggc ccaacttccc acaggatgga gagcctcaga 1320
gattgcacct ggagcctacc tggccacccc cacttagtgc ccccaggggc ccctaccact 1380
cctcagtggt gtctgccaca cggcccatgg tgatctctgc cactcgaccc acactacctt 1440
ctgcccacaa gacctctgtt atttcagcta cacgcccacc cctgagccct gtccacccac 1500
ctgccatggc ccctgccaca cctccagctg tgttctctga gcaccagatc cccaaaatca 1560
aggccaatta tccagacctg ccttttggcc acaagcctgg gataacctcg gccactcacc 1620
cagcacggtc tcctccgtac cagcccccca ttatctcaac caactatccc caagtcttcc 1680
ctccccacca ggcccctatg tctccagata cccacactat cacttatttg cctccagtcc 1740
cccctcacct tgatcctggg gataccactt ctaaagccca tcaacaccct ttgctcccag 1800
atgctccagg tatcagaacc caggcccccc agctttctgt ctcagctctc cagccccctc 1860
ttcctaccaa ctccaggtct tctgtccatg aaactcctgt gcctgctgcc aaccagcccc 1920
cagccttccc ttcttctccc ctcccccctc agaggcccac taaccagacc tcatctatca 1980
gccctacaca ttcctattcc agagcccctc tagtcccaag ggaaggagtt cccagtccca 2040
aatcagtgcc acagctgccc tcggtgccct ccacagcagc tccaacagcc ctggcagagt 2100
caggtcttgc aggccaaagc caaagggatg accgctggct gctggtggca ctcctggtgc 2160
caacatgtgt cttcttggtg gtgctgcttg ccctgggcat tgtgtactgc actcgctgtg 2220
gctcccacgc acccaacaag cggatcacgg actgctatcg ctgggtcaca catgctggga 2280
acaagagctc aacagaaccc atgcccccca gaggcagcct tacaggggta cagacctgta 2340
gaaccagtgt gggcggcagc ggcggcggcg gcagcggcgg caaaaatctg gattgttggg 2400
ttgataatga agaagatata gatgttatat taaaaaagag tacaatttta aatttagata 2460
ttaataatga tattatatca gatatatctg ggtttaattc atctgtaata acatatccag 2520
atgctcaatt ggtgcccgga ataaatggca aagcaataca tttagtaaac aatgaatctt 2580
ctgaagttat agtgcataaa gctatggata ttgaatataa tgatatgttt aataatttta 2640
ccgttagctt ttggttgagg gttcctaaag tatctgctag tcatttagaa caatatggca 2700
caaatgagta ttcaataatt agctctatga aaaaacatag tctatcaata ggatctggtt 2760
ggagtgtatc acttaaaggt aataacttaa tatggacttt aaaagattcc gcgggagaag 2820
ttagacaaat aacttttagg gatttacctg ataaatttaa tgcttattta gcaaataaat 2880
gggtttttat aactattact aatgatagat tatcttctgc taatttgtat ataaatggag 2940
tacttatggg aagtgcagaa attactggtt taggagctat tagagaggat aataatataa 3000
cattaaaact agatagatgt aataataata atcaatacgt ttctattgat aaatttagga 3060
tattttgcaa agcattaaat ccaaaagaga ttgaaaaatt atacacaagt tatttatcta 3120
taaccttttt aagagacttc tggggaaact gagctcgctt tcttgctgtc caatttctat 3180
taaaggttcc tttgttccct aagtccaact actaaactgg gggatattat gaagggcctt 3240
gagcatctgg attctgccta ataaaaaaca tttattttca ttgcgctcgc tttcttgctg 3300
tccaatttct attaaaggtt cctttgttcc ctaagtccaa ctactaaact gggggatatt 3360
atgaagggcc ttgagcatct ggattctgcc taataaaaaa catttatttt cattgcaaaa 3420
aaaaaaaaaa aaaaaaaaaa aaaaaaaaaa aaaaaaaaaa aaaaaaaaaa aaaaaaaaaa 3480
aaaaaaaaaa aaaaaaaaaa aaaaaaaaaa aaaaagagac g 3521
Sequence Number (ID): 20
Length: 15
Molecule Type: AA
Features Location/Qualifiers:
- source, 1 . . . 15
> mol type, protein
> organism, Synthetic construct
Residues:
HSSVLSVTRP VVVSA 15
Sequence Number (ID): 21
Length: 15
Molecule Type: AA
Features Location/Qualifiers:
- source, 1 . . . 15
> mol type, protein
> organism, Synthetic construct
Residues:
SVTRPVVVSA THPTL 15
Sequence Number (ID): 22
Length: 15
Molecule Type: AA
Features Location/Qualifiers:
- source, 1 . . . 15
> mol type, protein
> organism, Synthetic construct
Residues:
SRSPVSPAHQ ISVPA 15
Sequence Number (ID): 23
Length: 771
Molecule Type: DNA
Features Location/Qualifiers:
- source, 1 . . . 771
> mol type, other DNA
> organism, synthetic construct
Residues:
aaaaatctgg attgttgggt tgataatgaa gaagatatag atgttatatt aaaaaagagt 60
acaattttaa atttagatat taataatgat attatatcag atatatctgg gtttaattca 120
tctgtaataa catatccaga tgctcaattg gtgcccggaa taaatggcaa agcaatacat 180
ttagtaaaca atgaatcttc tgaagttata gtgcataaag ctatggatat tgaatataat 240
gatatgttta ataattttac cgttagcttt tggttgaggg ttcctaaagt atctgctagt 300
catttagaac aatatggcac aaatgagtat tcaataatta gctctatgaa aaaacatagt 360
ctatcaatag gatctggttg gagtgtatca cttaaaggta ataacttaat atggacttta 420
aaagattccg cgggagaagt tagacaaata acttttaggg atttacctga taaatttaat 480
gcttatttag caaataaatg ggtttttata actattacta atgatagatt atcttctgct 540
aatttgtata taaatggagt acttatggga agtgcagaaa ttactggttt aggagctatt 600
agagaggata ataatataac attaaaacta gatagatgta ataataataa tcaatacgtt 660
tctattgata aatttaggat attttgcaaa gcattaaatc cadaagagat tgaaaaatta 720
tacacaagtt atttatctat aaccttttta agagacttct ggggaaactg a 771
Sequence Number (ID): 24
Length: 3092
Molecule Type: DNA
Features Location/Qualifiers:
- source, 1 . . . 3092
> mol type, other DNA
> organism, synthetic construct
Residues:
taatacgact cactataggg gaaacccaaa catgggacgc tctaatacag acatggtgcg 60
aagagtctat tgagctagtt ggtagtcctc cggcccctga atgcggctaa tcctaactgc 120
ggagcacaca ccctcaagcc agagggcagt gtgtcgtaac gggcaactct gcagcggaac 180
cgactacttt gggtgtccgt gtttcatttt attcctatac tggctgctta tggtgacaat 240
tgagagatcg ttaccatata gctattggat tggccatccg gtgactaata gagctattat 300
atatcccttt gttgggttta taccacttag cttgaaagag gttaaaacat tacaattcat 360
tgttaagttg aatacagcaa aactagtgcc accatgctgc tgaggctgct gctggcctgg 420
gtggcagccg tgcctgctct cggccaggtg ccctggaccc ccgagcctag agcagcctgc 480
ggacccagca gctgctacgc cctgttcccc agaagaagga ccttcctgga agcctggcgc 540
gcctgcagag agctgggcgg caacctggcc acccccagaa cccccgagga agcccagagg 600
gtggacagcc tggtcggcgt gggacccgcc aacggactgc tgtggatcgg cctccagcga 660
caggccagac agtgccagcc ccagaggcca ctgaggggat tcatctggac caccggcgac 720
caggacaccg ccttcaccaa ctgggctcag cctgccaccg agggaccctg tcctgcccag 780
agatgtgccg ccctggaagc cagcggcgag cacagatggc tggaaggcag ctgcaccctg 840
gccgtggacg gctacctgtg ccagttcggc ttcgagggcg catgcccagc cctgcctctg 900
gaagtcggcc aggccggacc cgccgtgtac accaccccct tcaacctggt gtccagcgag 960
ttcgagtggc tgcccttcgg cagcgtggcc gctgtgcagt gtcaggccgg aagaggcgcc 1020
agcctgctgt gcgtgaagca gccctctggc ggcgtcggct ggtcccagac cggccctctg 1080
tgccctggca ccggctgcgg ccctgacaac ggcggctgcg agcacgagtg cgtggaggaa 1140
gtggacggcg ccgtgagctg caggtgcagc gagggcttca ggctggccgc cgacggccat 1200
agctgcgagg atccctgcgc acaggcccct tgcgagcagc agtgcgagcc tggcggccct 1260
cagggctaca gctgccactg caggctgggc ttcagacccg ccgaggacga cccccacaga 1320
tgcgtggaca ccgacgagtg ccagatcgcc ggcgtgtgcc agcagatgtg cgtgaactac 1380
gtgggcggct tcgagtgtta ctgctccgag ggccacgaac tcgaagccga tggcatctcc 1440
tgcagccccg ctggcgccat gggagcccag gctagccagg acctgaggga cgagctgctg 1500
gacgacggcg aggaaggcga ggacgaggaa gagccctggg aggacttcga cggcacctgg 1560
accgaggaac agggcatcct gtggctggcc cccacccacc cccctgactt cggcctgccc 1620
tacaggccca acttccccca ggacggcgag ccccagagac tgcacctgga acccacctgg 1680
ccccctcctc tgtctgcccc tagaggccct taccacagca gcgtggtgtc cgccaccagg 1740
cccatggtca tcagcgctac cagacctacc ctgcccagcg cccacaagac ctctgtgatt 1800
tccgccacta gacctcctct gagccccgtg cacccccctg ccatggcacc agccacacca 1860
cctgccgtgt tcagcgagca ccagatcccc aagatcaagg ccaactaccc cgacctgcct 1920
ttcggccaca agcccggcat caccagcgcc acccacccag ccagaagccc accctatcag 1980
ccacccatca tcagcaccaa ctaccctcag gtgttccccc cacaccaggc ccctatgagc 2040
cctgacaccc acaccatcac ctatctgcct cccgtgcctc ctcacctgga tcctggcgac 2100
accacctcca aggctcacca gcaccccctg ctgcctgacg cccctggcat caggacccag 2160
gccccccagc tgtccgtgag cgccctgcag cctcccctgc ccaccaacag caggtctagc 2220
gtgcacgaga cacctgtgcc tgccgccaac cagccccctg ccttccccag cagcccactg 2280
ccacctcaga ggcccaccaa ccagaccagc agcatcagcc ccacccacag ctacagcaga 2340
gcccccctgg tgcctcggga gggcgtgcct agccccaaga gcgtgcccca gctgcctagc 2400
gtgcccagca ccgccgctcc taccgccctg gccgaaagcg gcctggccgg acagtctcag 2460
agggacgacc ggtggctgct ggtcgccctg ctggtgccca cctgcgtgtt tctggtcgtg 2520
ctgctggcac tgggcatcgt gtactgcacc agatgcggca gccacgcccc caacaagagg 2580
atcaccgact gctacagatg ggtgacccac gccggcaaca agtccagcac cgagcccatg 2640
ccccccaggg gcagcctgac cggcgtgcag acctgcagga ccagcgtgtg atgatgaggg 2700
tttttaaaac agcctgtggg ttgatcccac ccacaggccc attgggcgct agcactctgg 2760
tatcacggta cctttgtgcg cctgttttat accccctccc ccaactgtaa cttagaagta 2820
acacacaccg atcaacagtc agcgtggcac accagccacg ttttgatcaa gcacttctgt 2880
taccccggac tgagtatcaa tagactgctc acgcggttga aggagaaagc gttcgttatc 2940
cggccaacta cttcgaaaaa cctagtaaca ccgtggaagt tgcagagtgt ttcgctcagc 3000
actaccccag tgtagatcag gtcgatgagt caccgcattc cccacgggcg accgtggcgg 3060
tggctgcgtt ggcggcctgc ccatgtttaa ac 3092
Sequence Number (ID): 25
Length: 3917
Molecule Type: DNA
Features Location/Qualifiers:
- source, 1 . . . 3917
> mol type, other DNA
> organism, synthetic construct
Residues:
taatacgact cactataggg gaaacccaaa catgggacgc tctaatacag acatggtgcg 60
aagagtctat tgagctagtt ggtagtcctc cggcccctga atgcggctaa tcctaactgc 120
ggagcacaca ccctcaagcc agagggcagt gtgtcgtaac gggcaactct gcagcggaac 180
cgactacttt gggtgtccgt gtttcatttt attcctatac tggctgctta tggtgacaat 240
tgagagatcg ttaccatata gctattggat tggccatccg gtgactaata gagctattat 300
atatcccttt gttgggttta taccacttag cttgaaagag gttaaaacat tacaattcat 360
tgttaagttg aatacagcaa aactagtgcc accatgctgc tgaggctgct gctggcctgg 420
gtggcagccg tgcctgctct cggccaggtg ccctggaccc ccgagcctag agcagcctgc 480
ggacccagca gctgctacgc cctgttcccc agaagaagga ccttcctgga agcctggcgc 540
gcctgcagag agctgggcgg caacctggcc acccccagaa cccccgagga agcccagagg 600
gtggacagcc tggtcggcgt gggacccgcc aacggactgc tgtggatcgg cctccagcga 660
caggccagac agtgccagcc ccagaggcca ctgaggggat tcatctggac caccggcgac 720
caggacaccg ccttcaccaa ctgggctcag cctgccaccg agggaccctg tcctgcccag 780
agatgtgccg ccctggaagc cagcggcgag cacagatggc tggaaggcag ctgcaccctg 840
gccgtggacg gctacctgtg ccagttcggc ttcgagggcg catgcccagc cctgcctctg 900
gaagtcggcc aggccggacc cgccgtgtac accaccccct tcaacctggt gtccagcgag 960
ttcgagtggc tgcccttcgg cagcgtggcc gctgtgcagt gtcaggccgg aagaggcgcc 1020
agcctgctgt gcgtgaagca gccctctggc ggcgtcggct ggtcccagac cggccctctg 1080
tgccctggca ccggctgcgg ccctgacaac ggcggctgcg agcacgagtg cgtggaggaa 1140
gtggacggcg ccgtgagctg caggtgcagc gagggcttca ggctggccgc cgacggccat 1200
agctgcgagg atccctgcgc acaggcccct tgcgagcagc agtgcgagcc tggcggccct 1260
cagggctaca gctgccactg caggctgggc ttcagacccg ccgaggacga cccccacaga 1320
tgcgtggaca ccgacgagtg ccagatcgcc ggcgtgtgcc agcagatgtg cgtgaactac 1380
gtgggcggct tcgagtgtta ctgctccgag ggccacgaac tcgaagccga tggcatctcc 1440
tgcagccccg ctggcgccat gggagcccag gctagccagg acctgaggga cgagctgctg 1500
gacgacggcg aggaaggcga ggacgaggaa gagccctggg aggacttcga cggcacctgg 1560
accgaggaac agggcatcct gtggctggcc cccacccacc cccctgactt cggcctgccc 1620
tacaggccca acttccccca ggacggcgag ccccagagac tgcacctgga acccacctgg 1680
ccccctcctc tgtctgcccc tagaggccct taccacagca gcgtggtgtc cgccaccagg 1740
cccatggtca tcagcgctac cagacctacc ctgcccagcg cccacaagac ctctgtgatt 1800
tccgccacta gacctcctct gagccccgtg cacccccctg ccatggcacc agccacacca 1860
cctgccgtgt tcagcgagca ccagatcccc aagatcaagg ccaactaccc cgacctgcct 1920
ttcggccaca agcccggcat caccagcgcc acccacccag ccagaagccc accctatcag 1980
ccacccatca tcagcaccaa ctaccctcag gtgttccccc cacaccaggc ccctatgagc 2040
cctgacaccc acaccatcac ctatctgcct cccgtgcctc ctcacctgga tcctggcgac 2100
accacctcca aggctcacca gcaccccctg ctgcctgacg cccctggcat caggacccag 2160
gccccccagc tgtccgtgag cgccctgcag cctcccctgc ccaccaacag caggtctagc 2220
gtgcacgaga cacctgtgcc tgccgccaac cagccccctg ccttccccag cagcccactg 2280
ccacctcaga ggcccaccaa ccagaccage agcatcagcc ccacccacag ctacagcaga 2340
gcccccctgg tgcctcggga gggcgtgcct agccccaaga gcgtgcccca gctgcctagc 2400
gtgcccagca ccgccgctcc taccgccctg gccgaaagcg gcctggccgg acagtctcag 2460
agggacgacc ggtggctgct ggtcgccctg ctggtgccca cctgcgtgtt tctggtcgtg 2520
ctgctggcac tgggcatcgt gtactgcacc agatgcggca gccacgcccc caacaagagg 2580
atcaccgact gctacagatg ggtgacccac gccggcaaca agtccagcac cgagcccatg 2640
ccccccaggg gcagcctgac cggcgtgcag acctgcagga ccagcgtggg atcctccaga 2700
agcaccccca tcccattcag ctacagcaag aacctggact gctgggtgga caacgaggag 2760
gacatcgacg tgatcctgaa gaagagcacc atcctgaacc tggacatcaa caacgacatc 2820
atcagcgaca tcagcggctt caacagcagc gtgatcacct accccgacgc ccagctggtg 2880
cccggcatca acggcaaggc catccacctg gtgaacaacg agagcagcga ggtgatcgtg 2940
cacaaggcca tggacatcga gtacaacgac atgttcaaca acttcaccgt gagcttctgg 3000
ctgagagtgc ctaaggtgag cgccagccac ctggagcagt acggcaccaa cgagtacagc 3060
atcatcagca gcatgaagaa gcacagcctg agcatcggca gcggctggag cgtgagcctg 3120
aagggcaaca acctcatctg gaccctgaag gatagcgccg gagaggtgag acagatcacc 3180
ttcagagacc tgcccgacaa gttcaatgcc tacctggcca acaagtgggt gttcatcacc 3240
atcaccaacg acagactgag cagcgccaac ctgtacatca acggcgtgct catgggcagc 3300
gccgagatca ccggcctggg cgccatcaga gaggacaaca acatcaccct gaagctggac 3360
agatgcaaca acaacaacca gtacgtgagc atcgacaagt tccggatctt ctgcaaggcc 3420
ctgaacccca aggagatcga gaagctgtac accagctacc tgagcatcac cttcctgaga 3480
gacttctggg gcaaccccct gagatacgac acctgatgat gagggttttt aaaacagcct 3540
gtgggttgat cccacccaca ggcccattgg gcgctagcac tctggtatca cggtaccttt 3600
gtgcgcctgt tttatacccc ctcccccaac tgtaacttag aagtaacaca caccgatcaa 3660
cagtcagcgt ggcacaccag ccacgttttg atcaagcact tctgttaccc cggactgagt 3720
atcaatagac tgctcacgcg gttgaaggag aaagcgttcg ttatccggcc aactacttcg 3780
aaaaacctag taacaccgtg gaagttgcag agtgtttcgc tcagcactac cccagtgtag 3840
atcaggtcga tgagtcaccg cattccccac gggcgaccgt ggcggtggct gcgttggcgg 3900
cctgcccatg tttaaac 3917
Sequence Number (ID): 26
Length: 22
Molecule Type: DNA
Features Location/Qualifiers:
- source, 1 . . . 22
> mol type, other DNA
> organism, Synthetic construct
Residues:
cctggaatca catcagctac tc 22
Sequence Number (ID): 27
Length: 20
Molecule Type: DNA
Features Location/Qualifiers:
- source, 1 . . . 20
> mol type, other DNA
> organism, Synthetic construct
Residues:
acgggtgcag tacacaatac 20
Sequence Number (ID): 28
Length: 20
Molecule Type: DNA
Features Location/Qualifiers:
- source, 1 . . . 20
> mol type, other DNA
> organism, Synthetic construct
Residues:
gaccaggata ccgcctttac 20
Sequence Number (ID): 29
Length: 20
Molecule Type: DNA
Features Location/Qualifiers:
- source, 1 . . . 20
> mol type, other DNA
> organism, Synthetic construct
Residues:
ccacctcttc cacacattca 20
Sequence Number (ID): 30
Length: 21
Molecule Type: DNA
Features Location/Qualifiers:
- source, 1 . . . 21
> mol type, other DNA
> organism, Synthetic construct
Residues:
ctcagttggt gccaggtata a 21
Sequence Number (ID): 31
Length: 22
Molecule Type: DNA
Features Location/Qualifiers:
- source, 1 . . . 22
> mol type, other DNA
> organism, Synthetic construct
Residues:
cctatccagc ttgagggtaa tg 22
Sequence Number (ID): 32
Length: 22
Molecule Type: DNA
Features Location/Qualifiers:
- source, 1 . . . 22
> mol type, other DNA
> organism, Synthetic construct
Residues:
cctgtgtcaa tttgggtttg ag 22
Sequence Number (ID): 33
Length: 23
Molecule Type: DNA
Features Location/Qualifiers:
- source, 1 . . . 23
> mol type, other DNA
> organism, Synthetic construct
Residues:
ctctccatcc tgtgggaaat tag 23

What is claimed is:

1. A composition for inducing an immune response against a tumor endothelial marker-1 (TEM1) protein, said composition comprising a delivery vehicle and a TEM1 mRNA that comprises a nucleotide sequence encoding a TEM1 protein or an immunogenic fragment thereof.

2. The composition of claim 1, wherein said delivery vehicle comprises a nanoparticle, a nanosome, a liposome, a biodegradable polymer complex, or a combination thereof.

3. The composition of claim 1, wherein said delivery vehicle comprises:
a lipid nanoparticle (LNP);
protamine;
a positively charged oil-in-water cationic nanoemulsion;
a chemically modified dendrimer complexed with a lipid;
cationic polymer comprising a lipid component;
polysaccharide; or
a cationic lipid, cholesterol, and optionally (i) a neutral lipid, (ii) a polyethylene-lipid, or (iii) a combination thereof.

4. The composition of claim 3, wherein said delivery vehicle comprises said lipid nanoparticle (LNP).

5. The composition of claim 4, wherein said LNP comprises cationic lipid, an ionizable cationic lipid, a sterol, a neutral lipid, a polyethylene-lipid, a polyethylene glycol (PEG)-modified lipid, a polymer-lipid conjugate, or a combination thereof.

6. The composition of claim 1 further comprising an adjuvant.

7. The composition of claim 6, wherein said adjuvant comprises a TT mRNA that is operatively linked to said TEM1 mRNA, and wherein said TT mRNA comprises a nucleotide sequence that encodes a tetanus toxoid or an immunogenic fragment thereof.

8. The composition of claim 7, wherein said TT mRNA has a nucleotide sequence having at least 80% identity to SEQ ID NO: 23 or a portion of SEQ ID NO: 23 that produces an immunogenic fragment of tetanus toxoid.

9. The composition of claim 7, wherein said TEM1 mRNA that is operatively linked to said TT mRNA encodes a polypeptide comprising TEM1 protein or an immunogenic fragment thereof that is operatively linked to N-terminal domain of fragment C of tetanus toxoid or an immunogenic fragment thereof.

10. The composition of claim 7, wherein said TEM1 mRNA that is operatively linked to said TT mRNA has a nucleotide sequence having at least 80% identity to SEQ ID NO: 5.

11. The composition of claim 1, wherein said TEM1 mRNA comprises a nucleic acid sequence that is at least 80% identity to SEQ ID NO: 2 or a portion of SEQ ID NO: 2 that produces said immunogenic fragment of TEM1 protein.

12. The composition of claim 1, wherein said TEM1 mRNA comprises a nucleotide sequence encoding said immunogenic fragment of TEM1 protein.

13. The composition of claim 12, wherein said immunogenic fragment of TEM1 protein comprises an amino acid sequence having at least 95% identity to amino acid residue 516-530, 511-525, 696-710, or a combination thereof of SEQ ID NO: 3.

14. The composition of claim 1, wherein said TEM1 mRNA comprises at least one modified nucleoside.

15. The composition of claim 14, wherein at least one uridine is replaced with said modified nucleoside.

16. The composition of claim 14, wherein said modified nucleoside is N1-methyl-pseudouridine (m1ψ).

17. A method for of inhibiting the growth of, decreasing the incidence, and/or recurrence of a tumor or a cancer cell in a subject, said method comprising administering to said subject a therapeutically effective amount of a composition of claim 1.

18. A method for treating a tumor or cancer in a subject comprising: administering to a subject in need of such a treatment a therapeutically effective amount of a composition of claim 1.

19. The method of claim 18, wherein said cancer or tumor comprises lung cancer or tumor, melanoma, breast cancer or tumor, kidney cancer or tumor, cervical cancer or tumor, head and neck cancer or tumor, breast cancer or tumor, an ano-genital cancer or tumor.

20. The method of claim 18, wherein said cancer comprises a melanoma, a sarcoma, a carcinoma, a lymphoma, a leukemia, a mesothelioma, a glioma, a germ cell tumor, a choriocarcinoma, a pancreatic cancer, an ovarian cancer, a gastric cancer, a carcinomatous lesion of the pancreas, pulmonary adenocarcinoma, colorectal adenocarcinoma, pulmonary squamous adenocarcinoma, gastric adenocarcinoma, an ovarian surface epithelial neoplasm (e.g., a benign, proliferative or malignant variety thereof), an oral squamous cell carcinoma, a non small-cell lung carcinoma, an endometrial carcinoma, a bladder cancer, a prostate carcinoma, an acute myelogenous leukemia (AML), a colon cancer, a lung cancer, a germ cell tumor, a uterine cancer, a thyroid cancer, a hepatocellular carcinoma, a liver cancer, a renal cancer, a Kaposi's sarcoma, or a sarcoma.


SEQUENCE LISTING (33 sequences)

SEQ ID NO: 1 - DNA, 2274 bp, Homo sapiens
  source 1..2274: mol_type = genomic DNA
atgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtga

SEQ ID NO: 2 - RNA, 2274 nt, Homo sapiens
  source 1..2274: mol_type = mRNA
atgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtga

SEQ ID NO: 3 - protein, 757 aa, Homo sapiens
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 4 - DNA, 3072 bp, synthetic construct
  source 1..3072: mol_type = other DNA
  gene 1..2274: gene = Human TEM1 DNA
  misc_feature 2275..2304: note = Linker
  gene 2305..3072: gene = Tetanus Toxoid
atgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgggcggcagcggcggcggcggcagcggcggcaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaactga

SEQ ID NO: 5 - RNA, 3072 nt, synthetic construct
  source 1..3072: mol_type = other RNA
  mRNA 1..2274: standard_name = Human TEM1 mRNA
  misc_structure 2275..2304: note = Linker
  mRNA 2305..3072: note = Tetanus Toxoid mRNA
atgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgggcggcagcggcggcggcggcagcggcggcaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaactga

SEQ ID NO: 6 - protein, 1023 aa, synthetic construct
  PEPTIDE 1..757: note = Human TEM1 Protein
  CHAIN 758..767: note = Linker
  PEPTIDE 768..1023: note = Tetanus Toxoid
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSVGGSGGGGSGGKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGN

SEQ ID NO: 7 - DNA, 2298 bp, Mus musculus
  source 1..2298: mol_type = genomic DNA
atgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgtga

SEQ ID NO: 8 - RNA, 2298 nt, Mus musculus
  source 1..2298: mol_type = mRNA
atgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgtga

SEQ ID NO: 9 - protein, 765 aa, Mus musculus
MLLRLLLAWVAAVPALGQVPWTPEPRAACGPSSCYALFPRRRTFLEAWRACRELGGNLATPRTPEEAQRVDSLVGVGPANGLLWIGLQRQARQCQPQRPLRGFIWTTGDQDTAFTNWAQPATEGPCPAQRCAALEASGEHRWLEGSCTLAVDGYLCQFGFEGACPALPLEVGQAGPAVYTTPFNLVSSEFEWLPFGSVAAVQCQAGRGASLLCVKQPSGGVGWSQTGPLCPGTGCGPDNGGCEHECVEEVDGAVSCRCSEGFRLAADGHSCEDPCAQAPCEQQCEPGGPQGYSCHCRLGFRPAEDDPHRCVDTDECQIAGVCQQMCVNYVGGFECYCSEGHELEADGISCSPAGAMGAQASQDLRDELLDDGEEGEDEEEPWEDFDGTWTEEQGILWLAPTHPPDFGLPYRPNFPQDGEPQRLHLEPTWPPPLSAPRGPYHSSVVSATRPMVISATRPTLPSAHKTSVISATRPPLSPVHPPAMAPATPPAVFSEHQIPKIKANYPDLPFGHKPGITSATHPARSPPYQPPIISTNYPQVFPPHQAPMSPDTHTITYLPPVPPHLDPGDTTSKAHQHPLLPDAPGIRTQAPQLSVSALQPPLPTNSRSSVHETPVPAANQPPAFPSSPLPPQRPTNQTSSISPTHSYSRAPLVPREGVPSPKSVPQLPSVPSTAAPTALAESGLAGQSQRDDRWLLVALLVPTCVFLVVLLALGIVYCTRCGSHAPNKRITDCYRWVTHAGNKSSTEPMPPRGSLTGVQTCRTSV

SEQ ID NO: 10 - DNA, 3096 bp, synthetic construct
  source 1..3096: mol_type = other DNA
  gene 1..2298: gene = Mouse TEM1
  misc_feature 2299..2328: note = Linker
  gene 2329..3096: gene = Tetanus Toxoid
atgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgggcggcagcggcggcggcggcagcggcggcaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaactga

SEQ ID NO: 11 - RNA, 3096 nt, Synthetic construct
  source 1..3096: mol_type = other RNA
  mRNA 1..2298: gene = Mouse TEM1 mRNA
  misc_feature 2299..2328: note = Linker
  gene 2329..3096: gene = Tetanus Toxoid mRNA
atgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgggcggcagcggcggcggcggcagcggcggcaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaactga

SEQ ID NO: 12 - protein, 1031 aa, Synthetic construct
  PEPTIDE 1..765: note = Mouse TEM1
  CHAIN 766..775: note = Linker
  PEPTIDE 776..1031: note = Tetanus Toxoid
MLLRLLLAWVAAVPALGQVPWTPEPRAACGPSSCYALFPRRRTFLEAWRACRELGGNLATPRTPEEAQRVDSLVGVGPANGLLWIGLQRQARQCQPQRPLRGFIWTTGDQDTAFTNWAQPATEGPCPAQRCAALEASGEHRWLEGSCTLAVDGYLCQFGFEGACPALPLEVGQAGPAVYTTPFNLVSSEFEWLPFGSVAAVQCQAGRGASLLCVKQPSGGVGWSQTGPLCPGTGCGPDNGGCEHECVEEVDGAVSCRCSEGFRLAADGHSCEDPCAQAPCEQQCEPGGPQGYSCHCRLGFRPAEDDPHRCVDTDECQIAGVCQQMCVNYVGGFECYCSEGHELEADGISCSPAGAMGAQASQDLRDELLDDGEEGEDEEEPWEDFDGTWTEEQGILWLAPTHPPDFGLPYRPNFPQDGEPQRLHLEPTWPPPLSAPRGPYHSSVVSATRPMVISATRPTLPSAHKTSVISATRPPLSPVHPPAMAPATPPAVFSEHQIPKIKANYPDLPFGHKPGITSATHPARSPPYQPPIISTNYPQVFPPHQAPMSPDTHTITYLPPVPPHLDPGDTTSKAHQHPLLPDAPGIRTQAPQLSVSALQPPLPTNSRSSVHETPVPAANQPPAFPSSPLPPQRPTNQTSSISPTHSYSRAPLVPREGVPSPKSVPQLPSVPSTAAPTALAESGLAGQSQRDDRWLLVALLVPTCVFLVVLLALGIVYCTRCGSHAPNKRITDCYRWVTHAGNKSSTEPMPPRGSLTGVQTCRTSVGGSGGGGSGGKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGN

SEQ ID NO: 13 - DNA, 56 bp, Synthetic construct
  source 1..56: mol_type = other DNA
acatttgcttctgacacaactgtgttcactagcaacctcaaacagacaccgccacc

SEQ ID NO: 14 - DNA, 264 bp, Synthetic construct
  source 1..264: mol_type = other DNA
gctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgc

SEQ ID NO: 15 - DNA, 105 bp, Synthetic construct
  source 1..105: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagacg

SEQ ID NO: 16 - DNA, 2699 bp, Synthetic construct
  source 1..2699: mol_type = other DNA
  5'UTR 1..56: note = 5'-UTR
  gene 57..2330: gene = hTEM1
  3'UTR 2331..2594: note = 3'-UTR
  polyA_site 2595..2699: note = Poly-A tail
acatttgcttctgacacaactgtgttcactagcaacctcaaacagacaccgccaccatgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtgagctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagacg

SEQ ID NO: 17 - DNA, 3497 bp, Synthetic construct
  source 1..3497: mol_type = other DNA
acatttgcttctgacacaactgtgttcactagcaacctcaaacagacaccgccaccatgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgggcggcagcggcggcggcggcagcggcggcaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaactgagctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagacg

SEQ ID NO: 18 - DNA, 2723 bp, Synthetic construct
  source 1..2723: mol_type = other DNA
acatttgcttctgacacaactgtgttcactagcaacctcaaacagacaccgccaccatgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgtgagctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagacg

SEQ ID NO: 19 - DNA, 3521 bp, Synthetic construct
  source 1..3521: mol_type = other DNA
acatttgcttctgacacaactgtgttcactagcaacctcaaacagacaccgccaccatgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgggcggcagcggcggcggcggcagcggcggcaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaactgagctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagacg

SEQ ID NO: 20 - protein, 15 aa, Synthetic construct
HSSVLSVTRPVVVSA

SEQ ID NO: 21 - protein, 15 aa, Synthetic construct
SVTRPVVVSATHPTL

SEQ ID NO: 22 - protein, 15 aa, Synthetic construct
SRSPVSPAHQISVPA

SEQ ID NO: 23 - DNA, 771 bp, synthetic construct
  source 1..771: mol_type = other DNA
aaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaactga

SEQ ID NO: 24 - DNA, 3092 bp, Synthetic construct
  source 1..3092: mol_type = other DNA
taatacgactcactataggggaaacccaaacatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaactagtgccaccatgctgctgaggctgctgctggcctgggtggcagccgtgcctgctctcggccaggtgccctggacccccgagcctagagcagcctgcggacccagcagctgctacgccctgttccccagaagaaggaccttcctggaagcctggcgcgcctgcagagagctgggcggcaacctggccacccccagaacccccgaggaagcccagagggtggacagcctggtcggcgtgggacccgccaacggactgctgtggatcggcctccagcgacaggccagacagtgccagccccagaggccactgaggggattcatctggaccaccggcgaccaggacaccgccttcaccaactgggctcagcctgccaccgagggaccctgtcctgcccagagatgtgccgccctggaagccagcggcgagcacagatggctggaaggcagctgcaccctggccgtggacggctacctgtgccagttcggcttcgagggcgcatgcccagccctgcctctggaagtcggccaggccggacccgccgtgtacaccacccccttcaacctggtgtccagcgagttcgagtggctgcccttcggcagcgtggccgctgtgcagtgtcaggccggaagaggcgccagcctgctgtgcgtgaagcagccctctggcggcgtcggctggtcccagaccggccctctgtgccctggcaccggctgcggccctgacaacggcggctgcgagcacgagtgcgtggaggaagtggacggcgccgtgagctgcaggtgcagcgagggcttcaggctggccgccgacggccatagctgcgaggatccctgcgcacaggccccttgcgagcagcagtgcgagcctggcggccctcagggctacagctgccactgcaggctgggcttcagacccgccgaggacgacccccacagatgcgtggacaccgacgagtgccagatcgccggcgtgtgccagcagatgtgcgtgaactacgtgggcggcttcgagtgttactgctccgagggccacgaactcgaagccgatggcatctcctgcagccccgctggcgccatgggagcccaggctagccaggacctgagggacgagctgctggacgacggcgaggaaggcgaggacgaggaagagccctgggaggacttcgacggcacctggaccgaggaacagggcatcctgtggctggcccccacccacccccctgacttcggcctgccctacaggcccaacttcccccaggacggcgagccccagagactgcacctggaacccacctggccccctcctctgtctgcccctagaggcccttaccacagcagcgtggtgtccgccaccaggcccatggtcatcagcgctaccagacctaccctgcccagcgcccacaagacctctgtgatttccgccactagacctcctctgagccccgtgcacccccctgccatggcaccagccacaccacctgccgtgttcagcgagcaccagatccccaagatcaaggccaactaccccgacctgcctttcggccacaagcccggcatcaccagcgccacccacccagccagaagcccaccctatcagccacccatcatcagcaccaactaccctcaggtgttccccccacaccaggcccctatgagccctgacacccacaccatcacctatctgcctcccgtgcctcctcacctggatcctggcgacaccacctccaaggctcaccagcaccccctgctgcctgacgcccctggcatcaggacccaggccccccagctgtccgtgagcgccctgcagcctcccctgcccaccaacagcaggtctagcgtgcacgagacacctgtgcctgccgccaaccagccccctgccttccccagcagcccactgccacctcagaggcccaccaaccagaccagcagcatcagccccacccacagctacagcagagcccccctggtgcctcgggagggcgtgcctagccccaagagcgtgccccagctgcctagcgtgcccagcaccgccgctcctaccgccctggccgaaagcggcctggccggacagtctcagagggacgaccggtggctgctggtcgccctgctggtgcccacctgcgtgtttctggtcgtgctgctggcactgggcatcgtgtactgcaccagatgcggcagccacgcccccaacaagaggatcaccgactgctacagatgggtgacccacgccggcaacaagtccagcaccgagcccatgccccccaggggcagcctgaccggcgtgcagacctgcaggaccagcgtgtgatgatgagggtttttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatgtttaaac

SEQ ID NO: 25 - DNA, 3917 bp, Synthetic construct
  source 1..3917: mol_type = other DNA
taatacgactcactataggggaaacccaaacatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaactagtgccaccatgctgctgaggctgctgctggcctgggtggcagccgtgcctgctctcggccaggtgccctggacccccgagcctagagcagcctgcggacccagcagctgctacgccctgttccccagaagaaggaccttcctggaagcctggcgcgcctgcagagagctgggcggcaacctggccacccccagaacccccgaggaagcccagagggtggacagcctggtcggcgtgggacccgccaacggactgctgtggatcggcctccagcgacaggccagacagtgccagccccagaggccactgaggggattcatctggaccaccggcgaccaggacaccgccttcaccaactgggctcagcctgccaccgagggaccctgtcctgcccagagatgtgccgccctggaagccagcggcgagcacagatggctggaaggcagctgcaccctggccgtggacggctacctgtgccagttcggcttcgagggcgcatgcccagccctgcctctggaagtcggccaggccggacccgccgtgtacaccacccccttcaacctggtgtccagcgagttcgagtggctgcccttcggcagcgtggccgctgtgcagtgtcaggccggaagaggcgccagcctgctgtgcgtgaagcagccctctggcggcgtcggctggtcccagaccggccctctgtgccctggcaccggctgcggccctgacaacggcggctgcgagcacgagtgcgtggaggaagtggacggcgccgtgagctgcaggtgcagcgagggcttcaggctggccgccgacggccatagctgcgaggatccctgcgcacaggccccttgcgagcagcagtgcgagcctggcggccctcagggctacagctgccactgcaggctgggcttcagacccgccgaggacgacccccacagatgcgtggacaccgacgagtgccagatcgccggcgtgtgccagcagatgtgcgtgaactacgtgggcggcttcgagtgttactgctccgagggccacgaactcgaagccgatggcatctcctgcagccccgctggcgccatgggagcccaggctagccaggacctgagggacgagctgctggacgacggcgaggaaggcgaggacgaggaagagccctgggaggacttcgacggcacctggaccgaggaacagggcatcctgtggctggcccccacccacccccctgacttcggcctgccctacaggcccaacttcccccaggacggcgagccccagagactgcacctggaacccacctggccccctcctctgtctgcccctagaggcccttaccacagcagcgtggtgtccgccaccaggcccatggtcatcagcgctaccagacctaccctgcccagcgcccacaagacctctgtgatttccgccactagacctcctctgagccccgtgcacccccctgccatggcaccagccacaccacctgccgtgttcagcgagcaccagatccccaagatcaaggccaactaccccgacctgcctttcggccacaagcccggcatcaccagcgccacccacccagccagaagcccaccctatcagccacccatcatcagcaccaactaccctcaggtgttccccccacaccaggcccctatgagccctgacacccacaccatcacctatctgcctcccgtgcctcctcacctggatcctggcgacaccacctccaaggctcaccagcaccccctgctgcctgacgcccctggcatcaggacccaggccccccagctgtccgtgagcgccctgcagcctcccctgcccaccaacagcaggtctagcgtgcacgagacacctgtgcctgccgccaaccagccccctgccttccccagcagcccactgccacctcagaggcccaccaaccagaccagcagcatcagccccacccacagctacagcagagcccccctggtgcctcgggagggcgtgcctagccccaagagcgtgccccagctgcctagcgtgcccagcaccgccgctcctaccgccctggccgaaagcggcctggccggacagtctcagagggacgaccggtggctgctggtcgccctgctggtgcccacctgcgtgtttctggtcgtgctgctggcactgggcatcgtgtactgcaccagatgcggcagccacgcccccaacaagaggatcaccgactgctacagatgggtgacccacgccggcaacaagtccagcaccgagcccatgccccccaggggcagcctgaccggcgtgcagacctgcaggaccagcgtgggatcctccagaagcacccccatcccattcagctacagcaagaacctggactgctgggtggacaacgaggaggacatcgacgtgatcctgaagaagagcaccatcctgaacctggacatcaacaacgacatcatcagcgacatcagcggcttcaacagcagcgtgatcacctaccccgacgcccagctggtgcccggcatcaacggcaaggccatccacctggtgaacaacgagagcagcgaggtgatcgtgcacaaggccatggacatcgagtacaacgacatgttcaacaacttcaccgtgagcttctggctgagagtgcctaaggtgagcgccagccacctggagcagtacggcaccaacgagtacagcatcatcagcagcatgaagaagcacagcctgagcatcggcagcggctggagcgtgagcctgaagggcaacaacctcatctggaccctgaaggatagcgccggagaggtgagacagatcaccttcagagacctgcccgacaagttcaatgcctacctggccaacaagtgggtgttcatcaccatcaccaacgacagactgagcagcgccaacctgtacatcaacggcgtgctcatgggcagcgccgagatcaccggcctgggcgccatcagagaggacaacaacatcaccctgaagctggacagatgcaacaacaacaaccagtacgtgagcatcgacaagttccggatcttctgcaaggccctgaaccccaaggagatcgagaagctgtacaccagctacctgagcatcaccttcctgagagacttctggggcaaccccctgagatacgacacctgatgatgagggtttttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatgtttaaac

SEQ ID NO: 26 - DNA, 22 bp, Synthetic construct
  source 1..22: mol_type = other DNA
cctggaatcacatcagctactc

SEQ ID NO: 27 - DNA, 20 bp, Synthetic construct
  source 1..20: mol_type = other DNA
acgggtgcagtacacaatac

SEQ ID NO: 28 - DNA, 20 bp, Synthetic construct
  source 1..20: mol_type = other DNA
gaccaggataccgcctttac

SEQ ID NO: 29 - DNA, 20 bp, Synthetic construct
  source 1..20: mol_type = other DNA
ccacctcttccacacattca

SEQ ID NO: 30 - DNA, 21 bp, Synthetic construct
  source 1..21: mol_type = other DNA
ctcagttggtgccaggtataa

SEQ ID NO: 31 - DNA, 22 bp, Synthetic construct
  source 1..22: mol_type = other DNA
cctatccagcttgagggtaatg

SEQ ID NO: 32 - DNA, 22 bp, Synthetic construct
  source 1..22: mol_type = other DNA
cctgtgtcaatttgggtttgag

SEQ ID NO: 33 - DNA, 23 bp, Synthetic construct
  source 1..23: mol_type = other DNA
ctctccatcctgtgggaaattag